(19) United States
(12) Patent Application Publication
(10) Pub. No.: US 2003/0078190 A1
(43) Pub. Date: Apr. 24, 2003

(54) Methods for tissue protection using highly effective inhibition of the renin-angiotensin system

(76) Inventor: Marc S. Weinberg, Seekonk, MA (US)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/155,824

(22) Filed: May 24, 2002

Related U.S. Application Data
(60) Provisional application No. 60/293,835, filed on May 25, 2001.

(51) Int. Cl. A61K 31/00

(52) U.S. Cl. 514/1

(57) ABSTRACT

Methods and pharmaceutical compositions are provided for protecting tissue of a subject from the effects of angiotensin II. The methods involve administering to subjects angiotensin receptor blockers (ARB), either by themselves at doses beyond those recommended or effective for the management of hypertension, or in combination with angiotensin-converting enzyme inhibitors (ACEI). The pharmaceutical compositions include both an ARB and an ACEI and are formulated in certain preferred embodiments for once-daily oral administration. The methods and pharmaceutical compositions are useful for the treatment of proteinuria, chronic or congestive heart failure, aneurysms, and vascular tissue hypertrophy.

### RELATED APPLICATION

This application claims benefit of U.S. provisional patent application Ser. No. 60/293,835, filed May 25, 2001.

### FIELD OF THE INVENTION

The invention pertains to pharmaceutical compositions and methods useful for the treatment of angiotensin II-mediated tissue effects which are distinct from hypertension.

### BACKGROUND OF THE INVENTION

The renin-angiotensin-aldosterone system (RAAS) is activated in hypertension, cardiac and renal disease and is attributed as a major factor in the morbidity and mortality associated with these disease syndromes.^{1,2} The beneficial effects of blocking the RAAS, using angiotensin-converting enzyme inhibitors (ACEI), is well supported by the number of approved indications in hypertension, chronic heart failure (CHF) and diabetic renal disease. Despite the proven effectiveness of angiotensin-converting enzyme (ACE) inhibition in cardiovascular disease there are inherent deficiencies in its ability to effectively block the RAAS. Numerous studies have demonstrated that long-term ACE inhibition results in incomplete RAAS blockade as plasma angiotensin II (AII) and aldosterone concentrations return to control levels, a phenomenon called ACE escape.

Incomplete RAAS blockade may explain the further progression in renal disease and heart failure (albeit at slower rates than with placebo) despite chronic ACE inhibition. It is now apparent that the activation of the RAAS not only affects circulatory regulation but also affects local tissue function (Table 1).

The circulatory response to RAAS activation is an acute elevation in blood pressure (BP), mediated through peripheral vasoconstriction and maintained by sodium and H₂O reabsorption. However, chronic stimulation of the RAAS leads to growth- and fibrosis-promoting processes at the tissue level which lead to further deleterious effects on end-organ function. The tissue-based RAAS is not acutely involved in BP regulation but rather on mitogenic effects of AII.

TABLE 1
Systemic (acute) and tissue-based (chronic) effects of RAAS activation^{6,7}

| Circulating RAAS | Tissue-based RAAS |
|---|---|
| Peripheral vasoconstriction | Cytokine activation |
| Stimulate release of aldosterone | Hypertrophy |
| Stimulate release of | Hyperplasia |
| arginine vasopressin | |
| Stimulate thirst and Na⁺ appetite | Remodeling |
| Renal Na⁺ and H₂O reabsorption | Fibrosis (e.g., collagen deposition) |

### SUMMARY OF THE INVENTION

These and further aspects related to the problems associated with angiotensin II-mediated tissue effects have now been discovered to be treated effectively using either (1) an angiotensin II receptor blocker (ARB) administered in doses higher than normally recommended or sufficient to control or reduce hypertension, or (2) an ARB administered in conjunction with administration of an ACEI. By using these higher doses or combinations, it has surprisingly been discovered that ARBs alone or ARBs in association with ACEIs exert beneficial effects at the tissue level, independent of their ability to control or further to reduce hypertension.

The invention describes experiments that demonstrate the effect of high dose ARBs, beyond Food and Drug Administration (FDA) recommended maximum doses and beyond doses effective for blood pressure control, on urinary protein excretion (proteinuria) in patients with heavy proteinuria, including nephrotic range (>3 g/d) proteinuria. The effective doses of ARB described herein are at least one-and-a-half times, and typically two or three to twenty times, the recommended maximum daily dose for control of hypertension. The tissue-protective effects at these high doses are shown to occur essentially without further reduction in blood pressure and without undue toxicity.

The invention also describes experiments that demonstrate the effect of combination treatment using ARB and ACEI in the treatment of a number of angiotensin II-related conditions and tissue effects, including aneurysms, excretion of protein into the urine (proteinuria), microalbuminuria, chronic or congestive heart failure (CHF), atherogenesis, atherosclerosis, tissue hypertrophy, and cytokine production.

Turning to specific aspects of the invention, in a first aspect the invention provides a method for treating an AII-mediated tissue effect in a subject. The method involves administering to a subject in need of treatment for an AII-mediated tissue effect an amount of an ARB effective for reducing an AII-mediated tissue effect in the subject, wherein the amount of ARB effective for reducing the AII-mediated tissue effect in the subject is more than an amount of the ARB that is effective for reducing or controlling blood pressure of the subject.

According to this aspect of the invention, in certain embodiments the amount of ARB effective for reducing an AII-mediated tissue effect is at least one-and-a-half times an amount effective for treatment or control of hypertension in the subject. In certain more preferred embodiments the amount of ARB effective for reducing an AII-mediated tissue effect is at least three times an amount effective for treatment or control of hypertension in the subject. Also in certain more preferred embodiments the amount of ARB effective for reducing an AII-mediated tissue effect is about three times to about twenty times an amount effective for treatment or control of hypertension in the subject.

Also according to this aspect of the invention, in certain embodiments the amount of ARB effective for reducing an AII-mediated tissue effect is at least one-and-a-half times a maximum daily dose recommended or approved for treatment or control of hypertension. In certain more preferred embodiments the amount of ARB effective for reducing an AII-mediated tissue effect is at least three times a maximum daily dose recommended or approved for treatment or control of hypertension. Also in certain more preferred embodiments the amount of ARB effective for reducing an AII-mediated tissue effect is about three times to about twenty times a maximum daily dose recommended or approved for treatment or control of hypertension.

In this and in all further aspects of the invention herein disclosed, in certain embodiments the AII-mediated tissue effect is an aneurysm. The aneurysm in some embodiments is an aortic aneurysm, including in certain embodiments an aortic root aneurysm.

In this and in all further aspects of the invention herein disclosed, in certain embodiments the AII-mediated tissue effect is proteinuria. In certain embodiments the proteinuria is microalbuminuria.

Also in this and in all further aspects of the invention herein disclosed, in certain embodiments the AII-mediated tissue effect is chronic or congestive heart failure (CHF).

In this and in all further aspects of the invention herein disclosed, in certain embodiments the AII-mediated tissue effect is atherogenesis or atherosclerosis. In certain preferred embodiments the atherosclerosis is associated with at least one condition selected from scleroderma, lupus erythematosus, rheumatoid arthritis, kidney disease, and solid organ transplantation.

Also in this and in all further aspects of the invention herein disclosed, in certain embodiments the AII-mediated tissue effect is tissue hypertrophy. In some embodiments the tissue hypertrophy is vascular tissue hypertrophy, for example cardiac (or, equivalently, myocardial or ventricular) hypertrophy.

In this and in all further aspects of the invention herein disclosed, in certain embodiments the AII-mediated tissue effect is cytokine production. The cytokine production inhibited by the methods herein can include that of transforming growth factor beta (TGF-β), fibroblast growth factor (FGF), and platelet-derived growth factor (PDGF). In a preferred embodiment the cytokine is transforming growth factor beta (TGF-β).

In certain embodiments of this first aspect of the invention, the administering involves administering a plurality of ARBs.

In this and in all further aspects of the invention herein disclosed, in certain embodiments the ARB is at least one compound selected from candesartan, eprosartan, irbesartan, losartan, olmesartan, telmisartan, valsartan, and prodrugs and salts thereof. This list is not to be understood to be limiting, since all ARBs, including those not currently approved for use in clinical practice in the United States, are also contemplated in this and all further aspects of the invention.

In this and in all further aspects of the invention herein disclosed, in certain preferred embodiments the ARB is at least one compound selected from candesartan, irbesartan, and prodrugs and salts thereof. In a preferred embodiment the ARB is candesartan cilexetil.

In certain embodiments of this first aspect of the invention, the method further involves administering to the subject at least one compound selected from aspirin, beta-blockers, aldactone, and compounds that can inhibit atherogenesis or platelet adhesion.

In certain embodiments of this first aspect of the invention, the method further involves administering to the subject at least one additional agent selected from diuretics, peripheral adrenergic blockers, central adrenergic stimulants, calcium channel blockers, vasodilators, and other antihypertensive agents excluding angiotensin-converting enzyme inhibitors.

In a second aspect the invention provides a method for treating an AII-mediated tissue effect in a normotensive subject. The method involves administering to a subject in need of treatment for an AII-mediated tissue effect an amount of an ARB effective for reducing an AII-mediated tissue effect in the subject, wherein the subject does not have hypertension, and wherein the amount of ARB effective for reducing an AII-mediated tissue effect in the subject is more than an amount of the ARB that is usually effective for reducing or controlling blood pressure in hypertensive subjects.

According to this aspect of the invention, in certain embodiments the amount of ARB effective for reducing an AII-mediated tissue effect is at least one-and-a-half times a maximum daily dose recommended or approved for treatment or control of hypertension. In certain more preferred embodiments the amount of ARB effective for reducing an AII-mediated tissue effect is at least three times a maximum daily dose recommended or approved for treatment or control of hypertension. Also in certain more preferred embodiments the amount of ARB effective for reducing an AII-mediated tissue effect is about three times to about twenty times a maximum daily dose recommended or approved for treatment or control of hypertension.

In certain embodiments of this second aspect of the invention, the administering involves a plurality of ARBs.

In certain embodiments of this second aspect of the invention, the method further involves administering to the subject at least one compound selected from aspirin, beta-blockers, aldactone, and compounds that can inhibit atherogenesis or platelet adhesion.

In certain embodiments of this second aspect of the invention, the method further involves administering to the subject at least one additional agent selected from diuretics, peripheral adrenergic blockers, central adrenergic stimulants, calcium channel blockers, vasodilators, and other antihypertensive agents excluding angiotensin-converting enzyme inhibitors.

In a third aspect the invention provides a method for treating an AII-mediated tissue effect in a subject. The method according to this aspect of the invention involves administering to a subject in need of treatment for an AII-mediated tissue effect an amount of ARB, and administering to the subject an amount of ACEI, wherein the amount of ARB and the amount of ACEI together is (1) effective for reducing the AII-mediated tissue effect in the subject and (2) more than an amount effective for achieving essentially a same degree of blood pressure reduction or blood pressure control in the subject. In one embodiment the method involves administering to a subject in need of treatment for an AII-mediated tissue effect an amount of ARB, and administering to the subject an amount of ACEI, wherein the amount of ARB and the amount of ACEI together are more effective for reducing the AII-mediated tissue effect in the subject than either the amount of ARB alone or the amount of ACEI alone. In one embodiment the method involves administering to a subject in need of treatment for an AII-mediated tissue effect an amount of ARB, and administering to the subject an amount of ACEI, wherein the amount of ARB and the amount of ACEI together is (1) effective for reducing the AII-mediated tissue effect in the subject and (2) more than is effective for reducing or controlling blood pressure of the subject.

According to this aspect of the invention, in certain embodiments the amount of ARB is more than an effective amount for achieving essentially the same degree of blood pressure reduction or blood pressure control in the subject.

Also according to this aspect of the invention, in certain embodiments the amount of ARB is at least one-and-a-half times an amount effective for treatment or control of hypertension in the subject. In certain more preferred embodiments the amount of ARB is at least three times an amount effective for treatment or control of hypertension in the subject. Also in certain more preferred embodiments the amount of ARB is about three times to about twenty times an amount effective for treatment or control of hypertension in the subject.

Also according to this aspect of the invention, in certain embodiments the amount of ARB is at least one-and-a-half times a maximum daily dose recommended or approved for treatment or control of hypertension. In certain more preferred embodiments the amount of ARB is at least three times a maximum daily dose recommended or approved for treatment or control of hypertension. Also in certain more preferred embodiments the amount of ARB is about three times to about twenty times a maximum daily dose recommended or approved for treatment or control of hypertension.

In certain embodiments of this third aspect of the invention, the administering an amount of ARB involves a plurality of ARBs.

In certain embodiments of this third aspect of the invention, the administering an amount of ACEI involves a plurality of ACEIs.

According to this aspect of the invention, in certain embodiments the ACEI is at least one compound selected from benazepril, captopril, enalapril, fosinopril, lisinopril, moexipril, perindopril, quinapril, ramipril, trandolapril, and prodrugs and salts thereof. This list is not to be understood to be limiting, since all ACEIs, including those not currently approved for use in clinical practice in the United States, are also contemplated in this aspect of the invention.

In certain embodiments of this third aspect of the invention, the method further involves administering to the subject at least one compound selected from aspirin, beta-blockers, aldactone, and compounds that can inhibit atherogenesis or platelet adhesion.

In certain embodiments of this third aspect of the invention, the method further involves administering to the subject at least one additional agent selected from diuretics, peripheral adrenergic blockers, central adrenergic stimulants, calcium channel blockers, and vasodilators.

In a fourth aspect the invention provides a method for treating an AII-mediated tissue effect in a normotensive subject. The method according to this aspect of the invention involves administering to a subject in need of treatment for an AII-mediated tissue effect an amount of ARB, and administering to the subject an amount of ACEI, wherein the subject does not have hypertension, and wherein the amount of ARB and the amount of ACEI together is (1) effective for reducing the AII-mediated tissue effect in the subject and (2) more than an amount that is usually effective for reducing or controlling blood pressure in hypertensive subjects.

Also according to this aspect of the invention, in certain embodiments the amount of ARB is at least one-and-a-half times a maximum daily dose recommended or approved for treatment or control of hypertension. In certain more preferred embodiments the amount of ARB is at least three times a maximum daily dose recommended or approved for treatment or control of hypertension. Also in certain more preferred embodiments the amount of ARB is about three times to about twenty times a maximum daily dose recommended or approved for treatment or control of hypertension.

In certain embodiments of this fourth aspect of the invention, the administering an amount of ARB involves a plurality of ARBs.

In certain embodiments of this fourth aspect of the invention, the administering an amount of ACEI involves a plurality of ACEIs.

According to this aspect of the invention, in certain embodiments the ACEI is at least one compound selected from benazepril, captopril, enalapril, fosinopril, lisinopril, moexipril, perindopril, quinapril, ramipril, trandolapril, and prodrugs and salts thereof. This list is not to be understood to be limiting, since all ACEIs, including those not currently approved for use in clinical practice in the United States, are also contemplated in this aspect of the invention.

In certain embodiments of this fourth aspect of the invention, the method further involves administering to the subject at least one compound selected from aspirin, beta-blockers, aldactone, and compounds that can inhibit atherogenesis or platelet adhesion.

In certain embodiments of this fourth aspect of the invention, the method further involves administering to the subject at least one additional agent selected from diuretics, peripheral adrenergic blockers, central adrenergic stimulants, calcium channel blockers, and vasodilators.

In a fifth aspect the invention provides a pharmaceutical composition for treating an AII-mediated tissue effect in a subject. The pharmaceutical composition according to this aspect of the invention includes an amount of ARB and an amount of ACEI, wherein the amount of ARB and the amount of ACEI together is (1) effective for reducing an AII-mediated tissue effect in a subject and (2) more than an amount that is usually effective for reducing or controlling blood pressure in hypertensive subjects.

In certain embodiments the pharmaceutical composition according to this aspect of the invention further includes a pharmaceutically acceptable carrier.

In certain embodiments of this fifth aspect of the invention, the amount of ARB is at least one-and-a-half times a maximum daily dose recommended or approved for treatment or control of hypertension. In certain more preferred embodiments of this fifth aspect of the invention, the amount of ARB is at least three times a maximum daily dose recommended or approved for treatment or control of hypertension. Also in certain more preferred embodiments of this aspect of the invention, the amount of ARB is about three times to about twenty times a maximum daily dose recommended or approved for treatment or control of hypertension.

In certain embodiments of this aspect of the invention, the amount of ARB involves a plurality of ARBs. As stated above, in certain preferred embodiments the ARB is at least one compound selected from candesartan, eprosartan, irbesartan, losartan, olmesartan, telmisartan, valsartan, and prodrugs and salts thereof. This list is not to be understood to be limiting, since all ARBs, including those not currently approved for use in clinical practice in the United States, are also contemplated in this aspect of the invention.

In certain embodiments of this fifth aspect of the invention, the amount of ACEI involves a plurality of ACEIs.

According to this aspect of the invention, in certain embodiments the ACEI is at least one compound selected from benazepril, captopril, enalapril, fosinopril, lisinopril, moexipril, perindopril, quinapril, ramipril, trandolapril, and prodrugs and salts thereof. This list is not to be understood to be limiting, since all ACEIs, including those not currently approved for use in clinical practice in the United States, are also contemplated in this aspect of the invention.

In certain embodiments of this fifth aspect of the invention, the pharmaceutical composition further includes at least one compound selected from aspirin, beta-blockers, aldactone, and compounds that can inhibit atherogenesis or platelet adhesion.

In certain embodiments of this fifth aspect of the invention, the pharmaceutical composition further includes at least one additional agent selected from diuretics, peripheral adrenergic blockers, central adrenergic stimulants, calcium channel blockers, and vasodilators.

In certain preferred embodiments of this aspect of the invention, the pharmaceutical composition is formulated for oral administration. In certain more preferred embodiments of this aspect of the invention, the pharmaceutical composition is formulated for once-daily administration. Also in certain more preferred embodiments of this aspect of the invention, the pharmaceutical composition is formulated for twice-daily administration.

In certain embodiments of this aspect of the invention, the pharmaceutical composition is formulated for parenteral administration.

These and other aspects of the invention are described below.

### BRIEF DESCRIPTION OF THE FIGURES

The following figures are provided for illustrative purposes only and are not required for understanding or practicing the invention.

FIG. 1 is a bar graph showing the effect of high-dose candesartan cilexetil (up to 96 mg/day) in reducing 24-hour urinary protein excretion (g/day). N=4-10 subjects per data point (see Table 4 for individual patient data). Data is presented as mean±SEM.

FIG. 2 is a bar graph showing the effect of progressive increases in the ACEI (lisinopril) dose on mean arterial pressure (MAP) and proteinuria in sixteen normotensive, proteinuric patients (>1.5 g/day) with IgA nephropathy. The ACEI was administered for four weeks at each dose with a three-week placebo period between doses. Adapted from Palla et al.³⁵

FIG. 3 is a diagram showing the renin-angiotensin-aldosterone system enzyme cascade depicting ACE and non-ACE pathways involved in the generation of AII. CAGE, chymostatin-sensitive AII-generating enzyme; t-PA, tissue plasminogen activator; NEP 24.11, neutral endopeptidase.

FIG. 4 is a bar graph showing the comparison of the blood pressure response between adding ARB to existing ACEI therapy or vice versa. Thirty hypertensive patients were randomized to receive either low dose losartan (25 mg) or ramipril (2.5) for four weeks. Thereafter, patients on the ACEI or the ARB either doubled the dosage of monotherapy or had low dose losartan or low dose ramipril, respectively, added to therapy. For the last four weeks of the study all patients, who were randomized to high dose monotherapy, had either high dose ramipril (5 mg) or high losartan (50 mg) added to therapy. Adapted from Bentivoglio et al.¹⁵¹

FIG. 5 is a graph showing the time course of sitting diastolic blood pressure in 244 patients undergoing long-term open-label treatment with 16 mg of candesartan cilexetil. Adapted from Sever et al.¹⁶³

FIG. 6 is a bar graph showing the effect of increasing the dose of losartan (50 to 100 mg) or enalapril (10 to 20 mg) in 16 type-1 diabetic patients on mean arterial pressure (MAP) and proteinuria. Patients were randomized to treatment periods that lasted two months for each dose. Adapted from Andersen et al.¹⁸⁵

FIG. 7 is a bar graph showing the antiproteinuric effect of the combination of losartan (LOS; 50 mg) with an ACEI, compared to monotherapy with LOS or ACEI, in eight normotensive patients with IgA nephropathy and non-nephrotic proteinuria (1-3 g/day). Urinary protein excretion was measured at the end of each four-week period for ACEI monotherapy, ACEI+LOS combination therapy, LOS monotherapy, and LOS+ACEI combination therapy. *p<0.05 vs. baseline; #p<0.05 vs. ACEI or losartan monotherapy. Adapted from Russo et al.¹⁹⁴

FIG. 8 is a bar graph showing the change in aortic root size in 15 patients receiving maximum or above-maximum recommended doses of the ARB candesartan for at least nine months.

### DETAILED DESCRIPTION OF THE INVENTION

In order to facilitate describing various aspects of the invention in more detail, some definitions are set forth below.

The invention describes methods and compositions useful to inhibit the RAAS and that are directed to blocking the short-term (circulating) and long-term (tissue-based) effects of RAAS stimulation.

An angiotensin-converting enzyme inhibitor (ACEI) is a pharmaceutical agent that inhibits the enzymatic activity of angiotensin-converting enzyme. A number of ACEI are approved and available for clinical use in the treatment of hypertension, chronic or congestive heart failure (CHF), and post-myocardial infarction use. Doses used for the treatment or control of hypertension generally exceed those used for other indications. It is widely recognized that the antihypertensive effect of ACEI is non-linear, i.e., approaches a plateau with increasing dosage. Representative ACEI in use in the United States are presented in Table 2. Other ACEI, including those in clinical use outside the United States and those in development, are also embraced by the invention.

The predominant actions of ACEI are to block the activity of the RAAS by inhibiting both the circulating as well as the tissue effects of AII. Simply increasing the dose of the ACEI to higher levels has resulted in further inhibition of the RAAS; however, the optimal ACEI dose to maximally block the RAAS is not known and has not been studied. The tissue-based RAAS is known to be more difficult to block, due to difficulties in tissue penetration and to the existence of tissue-based enzymes, other than ACE, that are involved in the generation of AII and may account for the inability to maintain adequate RAAS blockade over time while using ACEI therapy.⁹ Thus, strategies are needed to improve RAAS blockade while on chronic ACE inhibition, and these should be targeted to more effective blockade of the tissue RAAS.

TABLE 2
ACE Inhibitors Approved for Clinical Use in the United States

| | | | | Max. |
|---|---|---|---|---|
| Generic | | | Usual Dose¹ | Dose² |
| name | Trade Name | Supplier | mg/d | mg/d |
| benazepril | LOTENSIN | Novartis | 20-40 | 80 |
| captopril | | Mylan | 150 | 450 |
| enalapril | VASOTEC ® | Merck | 10-40 | 40 |
| fosinopril | MONOPRIL | Bristol-Myers | 20-40 | 80 |
| | | Squibb | | |
| lisinopril | PRINIVIL ® | Merck | 20-40 | 80 |
| lisinopril | ZESTRIL | AstraZeneca | 20-40 | 80 |
| moexipril | UNIVASC | Schwarz | 7.5-30 | 60 |
| perindopril | ACEON ® | Solvay | 4-8 | 16 |
| quinapril | ACCUPRIL ® | Parke-Davis | 20-80 | 80 |
| ramipril | ALTACE | Monarch | 2.5-20 | 20 |
| trandolapril | MAVIK | Knoll | 2-4 | 8 |

¹Usual Dose refers to the usual maintenance dose recommended for the treatment of hypertension in humans.
²Max. Dose refers to the recommended maximum daily dose in humans.

An angiotensin II receptor blocker (ARB) is a pharmaceutical agent that selectively blocks the binding of AII to the AT₁ receptor found in many tissues. These provide the potential for more complete blockade of the RAAS by being able to prevent the binding of AII to its primary biological receptor (AII type 1 receptor [AT₁]). The apparent low rate of side-effects when using ARBs (i.e., side-effects similar to placebo rates) has given this new drug class a special and unique benefit over all other RAAS blockers.^{10,11} Representative ARBs in use in the United States are presented in Table 3. Other ARBs, including those in clinical use outside the United States and those in development, are also embraced by the invention.

An AII-mediated tissue effect refers to a direct or indirect effect of AII on a living tissue, including without limitation cytokine production, elastin and collagen production, hypertrophy, growth, angiogenesis and atherogenesis. These effects can also include, for example, development and expansion of an aneurysm of the aorta or other blood vessel or vascular tissue, proteinuria, microalbuminuria, chronic or congestive heart failure, and secretion of cytokines, including but not limited to transforming growth factor beta-1 (TGF-β1), fibroblast growth factor (FGF), and platelet-derived growth factor (PDGF) which promote extracellular matrix accumulation (e.g., fibronectin, type I, IlI and IV collagen deposition and osteopontin) leading to fibrotic changes.^{55,58}.

TABLE 3
ARBs Approved for Clinical Use in the United States

| | | | | Max. |
|---|---|---|---|---|
| | | | Usual Dose¹ | Dose² |
| Generic Name | Trade Name | Supplier | mg/d | mg/d |
| candesartan | ATACAND ® | AstraZeneca | 8-32 | 32 |
| | | LP | | |
| eprosartan | TEVETEN ® | Unimed | 400-800 | 800 |
| irbesartan | AVAPRO ® | Bristol-Myers | 150 | 300 |
| | | Squibb | | |
| losartan | COZAAR ® | Merck | 50 | 100 |
| olmesartan | BENICAR ™ | Sankyo Pharma | 20-40 | 40 |
| telmisartan | MICARDIS ® | Boehringer | 20-80 | 80 |
| | | Ingelheim | | |
| valsartan | DIOVAN ® | Novartis | 80-320 | 320 |

¹Usual Dose refers to the usual maintenance dose recommended for the treatment of hypertension in humans.
²Max. Dose refers to the recommended maximum daily dose in humans.

A subject in need of treatment for an AII-mediated tissue effect is a subject with at least one identifiable sign, symptom, or laboratory finding sufficient to make a diagnosis of an AII-mediated tissue effect, made in accordance with clinical or laboratory standards known in the art for identifying such an effect. The diagnosis may use any method suitable for making the clinical or laboratory diagnosis. For example, the diagnosis of an aneurysm may be made by any one or combination of physical examination, X-ray examination, including axial tomography, magnetic resonance imaging, or angiography performed with or without contrast, ultrasound examination. As another example, a diagnosis of an AII-mediated tissue effect as herein defined may include measurement of circulating or local concentrations of certain cytokines, as can be performed by methods which may include enzyme-linked immunosorbent assay (ELISA), bioactivity, etc.

An “effective amount” refers to that amount of a compound which, when administered to a subject, prevents the onset of, alleviates the symptoms of, or stops the progression of a disorder or condition being treated in the subject. Thus for example an amount of an ARB or an amount of a combination of an ARB and ACEI that is effective for treating or reducing an AII-mediated tissue effect is an amount that prevents the onset of, alleviates the symptoms of, or stops the progression of an AII-mediated tissue effect. In some embodiments an effective amount is that amount that reduces a continuously variable parameter; such a continuously variable parameter may include, for example, a concentration of protein excreted in the urine, blood pressure, or diameter of an aneurysm.

The term “treating” as used herein refers to administering to a subject an effective amount of a compound that is sufficient to prevent the onset of, alleviate the symptoms of, or stop the progression of a disorder or condition being treated in the subject.

The term “subject” as used herein refers to a mammal. In a preferred embodiment a subject is a human.

Pharmaceutical compositions of the instant invention will be described further below.

The primary rationale for using ACEI and ARBs in combination is to achieve a more specific and complete RAAS blockade by inducing blockade at different sites of the AII generation pathway. Furthermore, the counter-regulatory responses to RAAS blockade, using ACEI or ARBs, will be antagonized by combined therapy.12,13 In this way, the elevation in plasma angiotensin I (Ang I) levels on an ACEI would not be able to overcome RAAS blockade, as an ARB would block all actions of AII generated via ACE or non-ACE pathways. The ACEI-induced fall in plasma AII would also help to counteract the rise in AII which could stimulate tissue angiotensin (AT₁ and AT₂) receptors not fully blocked.¹⁴

Therefore, inhibition of the RAAS, using ACEI and ARBs in combination, is more effective than with either agent alone, as recent studies have reported greater increases in plasma renin activity (PRA) with the combination compared with ACE inhibition alone.¹⁵⁻¹⁷

Theoretically, the greater the RAAS blockade, the greater the attenuation of deleterious RAAS-induced local tissue trophic effects. As a consequence, long-term target organ protection occurs as a specific goal of combined RAAS therapy.

The proposed limitations of ACEI monotherapy in cardiovascular disease are related to its inability to effectively block the RAAS. The ACE escape effect, associated with the use of ACEI, occurs by either incomplete ACE inhibition or the generation of AII by ACE-independent enzymatic pathways. Additionally, ACEI may not fully inhibit the actions of the local or tissue-based RAAS. The use of ARBs, which block the direct effect of AII at the AT₁ receptor, when added to ACEI, can ensure more complete suppression of the RAAS.

Clinical studies have shown that, within the therapeutic dose range for ACEI, plasma AII and aldosterone levels gradually return to control levels.^{3,18-21} One possible mechanism for the ACE escape phenomenon with chronic ACEI therapy may be attributed to the reactive rise in PRA and Ang I levels.^{22,23} The body's counter-regulatory response to an ACEI (i.e., hyperreninemia) may, in some patients, reach high enough levels to overcome the enzyme inhibition, thus causing increases in AII.^{4,24} The acute reduction in plasma AII levels following ACE inhibition attenuates the persistent negative feedback response on the renin-producing juxtaglomerular cells. Thus, the inhibitory signal for renin release is removed following ACE inhibition, resulting in elevated PRA and Ang I levels. Elevations in PRA and Ang I levels as great as 2-3 times above normal may be sufficient to overcome enzyme inhibition.^{18,21} Non-sustained ACE inhibition, for the complete 24 hours (e.g., plasma half-life differences between enalapril and lisinopril), may also be responsible for increases in plasma AII levels.

During chronic ACE inhibition, an effective means to maintain efficient RAAS blockade is to escalate the ACEI dose.¹³ The maximum, sustained antihypertensive response to ACE inhibition is only achieved by using higher doses than typically recommended for BP control.^{25,26} More effective and maximum long-term BP control can be achieved by using higher doses for benazepril (up to 80 mg/day),²⁶ enalapril (up to 40 mg/day),²⁷ captopril (up to 150 mg/day),²⁸ lisinopril (up to 80 mg/day),²⁹ ramipril (up to 20 mg/day),³⁰ and quinapril (up to 80 mg/day).³¹ Further, for patients with heart failure or renal disease, the antihypertensive dosage used for ACE inhibition is typically lower than the optimal recommended doses.²⁶ Increased doses of the ACEI have also been reported to have beneficial effects in patients with CHF and renal disease, suggesting more effective long-term blockade of the RAAS.³²⁻³⁵

Increases in the ACEI dose, above which no further antihypertensive response is observed, results in continual increases in PRA and, therefore, a more complete RAAS blockade.³⁶⁻³⁷ Blockade of the tissue-based RAAS, which requires higher doses of ACEI or ARBs, is important for optimal target organ protection. Using the low or recommended antihypertensive dose for the ACEI may be effective in blocking the circulating RAAS but may have little effect on the tissue-based RAAS, and thus only a limited long-term effect in target organ disease protection.³⁸

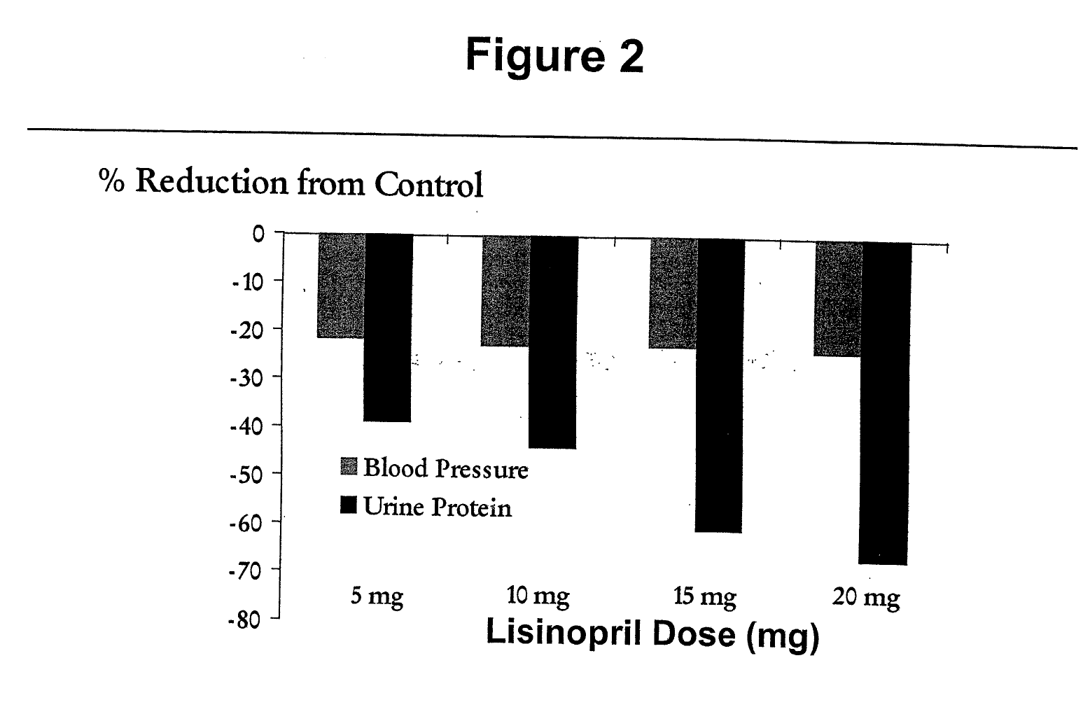

Incomplete ACE inhibition would suggest that the antihypertensive and hemodynamic effects of ACEI therapy would be short-lived.²¹ However, there have been no clinical reports of reduced BP lowering or hemodynamic efficacy with chronic ACEI therapy.²² Rather, incomplete ACE inhibition may be more related to reduced inhibition of the tissue-based RAAS.³⁹ A study by Palla et al.³⁵ reported that increasing the dose of lisinopril resulted in additional renal protective effects, independent of any BP reductions (FIG. 2).³⁵ To compensate for incomplete ACE inhibition, more effective and complete RAAS blockade can be achieved by increasing the dose of the ACEI or by adding another RAAS blocker, such as an ARB.¹³ The effect of one of these two strategies will not necessarily result in further lowering of BP but will result in greater inhibition of the tissue RAAS.⁴⁰

Non-ACE Pathways for the Generation of AII

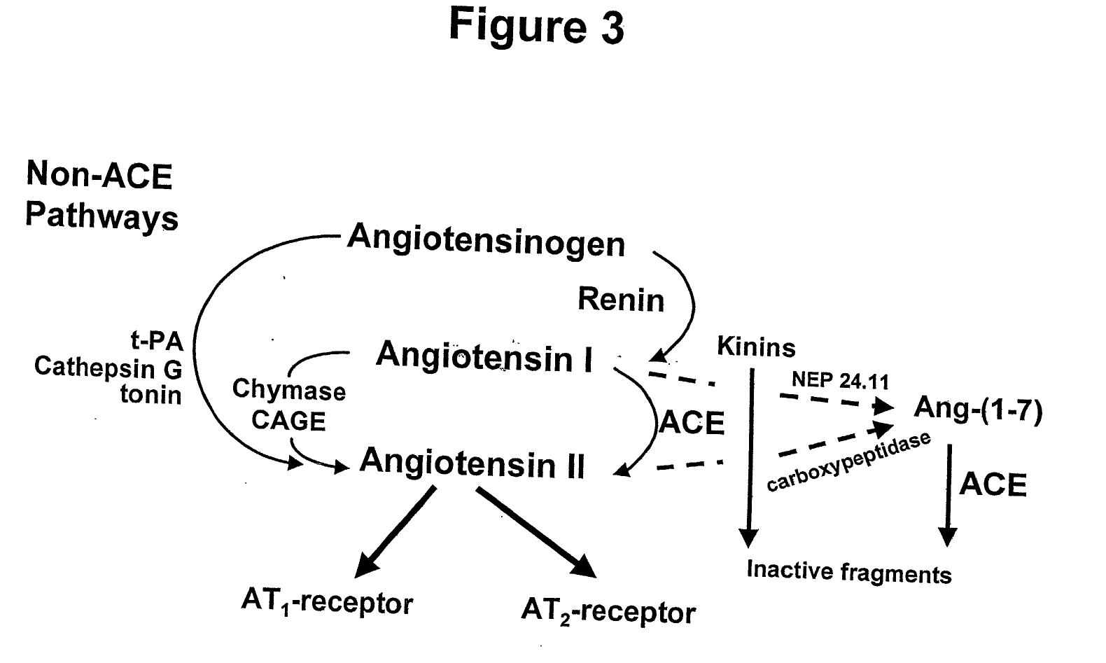

Ineffective RAAS blockade while on ACEI therapy may be due to AII generation by activation of other enzyme systems. Recent studies have reported the existence of other enzymatic pathways that can form AII, independent of ACE, that are not blocked by ACEI (FIG. 3).⁴¹⁻⁴⁵ AII can be formed through the action of serine proteases (e.g., chymase) which are localized to the interstitium of a variety of tissues, particularly cardiac, vascular and renal tissues.⁴⁶ In human heart tissue, chynase has been identified as the major AII-forming enzyme. Urata et al.⁴⁷ reported that in the heart, up to 70% of locally generated AII may be through chymase, whereas in the kidney, it is approximately 20%.⁴⁸ Hollenberg⁴² recently reported that non-ACE pathways account for 30-40% of AII formed in the kidney in diabetics. Thus, the formation of AII through non-ACE pathways contributes substantially to the tissue-based RAAS while not contributing directly to the circulating RAAS.

The lack of information pertaining to the stimulation and/or inhibition of these serine proteases, makes it nearly impossible to determine the functional significance of these alternative AII-forming pathways. However, it can be speculated that they play important roles in local cell function and cell growth and are not thought to contribute to important systemic (i.e., circulating) cardiovascular effects. Furthermore, some investigators have speculated that, rather than playing an important role in normal cellular function, non-ACE pathways are activated in syndromes of disease where there are high levels of oxidative stress, such as vascular pro-inflammatory and atherogenic processes.^{43,49} Furthermore, when plasma renin activity is low, the tissue RAAS may still be active through local AII production, via the enzymatic action of chymase.⁵⁰

Previously, some investigators have failed to identify these non-ACE pathways as being clinically significant for the generation of AII. Most animal studies have failed to demonstrate important differences between the tissue-protective effects of ARBs compared with ACEI. However, more recently it has been reported that species-related differences in non-ACE pathways generation of AII can explain inconsistent results between various experimental animal models.⁴⁴ In rats, AII is generated entirely through ACE and does not involve any of the non-ACE pathways, whereas in humans, monkeys, dogs, and hamsters, chymase does generate AII.^{43,44} Thus, rat studies reporting equivalence between ACEI and ARBs for cardiac and renal protection may not be applicable to man and are therefore not a good model to study differences between ACEI and ARBS.^{45,46}

If the ACE escape response is entirely due to the counter-regulatory response to ACE inhibition ultimately overcoming enzyme inhibition through mass action, then there is no need to invoke other mechanisms, such as AII formation through alternative pathways.²⁴ However, if, in response to ACE inhibition, elevations in both PRA and Ang I stimulate the activity of these non-ACE pathways, then non-ACE pathways may account for the reduced long-term cardiac and renal protective effects of ACEI.^{51,52} Accordingly, in certain aspects of the instant invention, blocking both ACE and non-ACE pathways responsible for the generation of AII is believed to be an important therapeutic goal for optimal cardiovascular protection.

Tissue RAAS

There is direct evidence supporting the existence and functional importance of a tissue-based RAAS at a variety of sites (e.g., blood vessels, heart, kidney).⁵³ Activation of the tissue RAAS accounts for the long-term effects (e.g., vascular remodeling, glomerular hypertrophy, left ventricular hypertrophy, angiogenesis) of AII (Table 1).^{8,9} The endocrine or circulating RAAS is more focused on hemodynamic compensatory responses to maintain plasma flow and BP homeostasis.⁸ The tissue-based RAAS functions as a regulator of local AII activity, such that it is possible to have increased tissue RAAS activity without any detectable changes in plasma RAAS activity.^{42,54-55}

Blockade of the RAAS occurs through inhibition of AII in both plasma and tissues. Effective blockade of the circulating RAAS occurs at much lower doses than those necessary to effectively block the tissue RAAS.²⁴ Thus, the tissue-based RAAS functions independently from the peripheral circulation but is more difficult to block completely.^{9,56} Incomplete tissue RAAS blockade while on an ACEI may be due to the difficulties in penetrating various tissues with organ-specific differences and/or to the existence of non-ACE pathways generating AII. An advantage of ARB therapy, with respect to tissue RAAS blockade, is its ability to block the effects of AII at the AT₁ receptor regardless of whether AII is generated via ACE or non-ACE pathways.

Chronic elevations in plasma and tissue AII concentrations stimulate cardiac, vascular and renal mitogenic processes, partly mediated through elevations in cytokines. Increases in AII will stimulate the release of cytokines such as transforming growth factor beta-1 (TGF-β1), fibroblast growth factor (FGF), and platelet-derived growth factor (PDGF) which promote extracellular matrix accumulation (e.g., fibronectin, type I, III and IV collagen deposition and osteopontin) leading to fibrotic changes.^{55,58} Evidence suggests that inhibition of cytokine expression is a primary mechanism by which RAAS blockade prevents progressive glomerular, vascular and cardiac disease.^{59,60} Both ARBs and ACEI, through similar mechanisms, prevent the undesirable growth-promoting effects of AII through indirect (hemodynamically mediated) and direct inhibition of cytokine expression. However, since cytokine stimulation affects tissue mitogenic processes, higher doses of RAAS blockers are required to suppress cytokine expression effectively.¹⁰ The degree of reduction of cytokine expression can been used as an index of the extent of tissue RAAS inhibition.⁵⁸ Due to the limitations in tissue RAAS blockade by ACEI, blockade may be more effectively accomplished by adding an ARB to existing ACEI therapy without trying to titrate the ACEI dose to some undefined optimal level.

AII Type-2 Receptor

There are multiple receptors for AII, but only two receptors with biological effects that are known, the AT₁ and AT₂ receptors (FIG. 3). Stimulation of the AT₁ receptor results in the characteristic effects of AII, vasoconstriction, renal Na⁺ reabsorption and cell proliferation. The AT₂ receptor, which is of secondary importance to the AT₁ receptor, was originally thought to be important only for fetal growth and development. However, more recent data has suggested that the AT₂ receptor may have the important role of modulating the effects of chronic AT₁ receptor stimulation. Stimulation of the AT₂ receptor by AII in the adult results in vasodilation and cell growth inhibition.⁶³ Thus, the AT₂ receptor-mediated effects are inhibitory to AT₁ receptor-mediated mitogen-induced growth effects, indicating a balancing mechanism for AII-controlled mechanisms.⁶⁴

In clinical syndromes of cardiovascular disease, such as ventricular remodeling and myocardial ischemia, the AT₂ receptor has been reported to be re-expressed or up-regulated.^{65,66} Stimulation of the AT₂ receptor may control excessive growth mediated, in part, by AT₁ receptors.⁶⁴ The cardiovascular effects of AT₂ receptor stimulation are primarily of local benefit, mediated through increases in nitric oxide (NO) and other local vasodilator, anti-mitogenic substances, contributing to the tissue-protective effects of ARBS.^{67,68} This turning on of AT₂ receptors could serve to counterbalance or modulate excessive effects of AT₁ receptor stimulation in cardiovascular disease. However, despite the supposed benefits of chronic AT₂ receptor stimulation by ARBs, the combination of an ACEI and ARB would act to reduce plasma AII levels, removing this theoretical advantage.^{13,69}

Rationale for Maintaining ACEI Therapy When Adding an ARB

The benefits of ACEI therapy in hypertension, cardiac and renal disease are well-founded. The notion of adding new pharmacological treatments to existing ACEI therapy is much more acceptable than that of replacing existing ACEI therapy, particularly considering the reported benefits of ACE inhibition. It could be argued that the theoretical benefits and advantages of ARBs outweigh the advantages of ACEI therapy. However, the potential advantages of ARBs over ACEI have not yet been proven in randomised clinical trials. Until those studies are completed, ARBs will not replace ACEI therapy but will be used in patients who are ACEI-intolerant or as add-on therapy to ACEI, primarily in cardiac and renal disease.

One of the reasons for maintaining patients on ACEI therapy when combining ARBs is related to the ability to prevent the counter-regulatory responses to AT₁ receptor blockade. ACE inhibition will prevent the increase in plasma AII levels, thereby reducing the competition between endogenous AII and the ARB for the receptor site. Additionally, and perhaps more importantly, the ability of ACEI to block tissue-ACE activity and to potentiate local vasodilator and antiproliferative substances (i.e., bradykinin and angiotensin peptide fragment, Ang-[1-7]) may increase the tissue-protective effects of therapy.

Reduction of Plasma AII Levels

Adding an ACEI to ARB therapy reduces the formation of AII and thus reduces the competition at the AT₁ receptor between vasoactive hormone (AI) and drug (ARB). Following the administration of an ACEI, there is an acute reduction in plasma AII concentrations. However, recent studies have reported that chronic ACE inhibition results in plasma AII levels returning to control levels over a period of weeks to months.^{20,70,71} It is not known if chronic ACEI therapy, when combined with an ARB, will still result in elevation of plasma AII levels over time due to the ACE escape effect. It is well appreciated that the therapeutic efficacy of most ARBs is not affected by elevated plasma AII levels. Thus, it is believed by the applicant that the benefit of reducing plasma AII levels, while on an ARB, may be more important for lower affinity ARBs such as losartan, where competition at the AT₁ receptor reduces their therapeutic effect.^{72,73} Elevations in plasma AII levels while on an ARB will result in the production of angiotensin IV (Ang IV), a byproduct of aminopeptidase-induced AII metabolism.⁷⁴ Ang IV (Ang-3-8) is believed to have its own receptor and to stimulate the expression of plasminogen-activator inhibitor 1 (PAI-1) in the vascular endothelium.⁷⁵ Stimulation of PAI-1, through increases in plasma Ang IV levels, is pro-thrombotic, as it inhibits tissue plasminogen activator (t-PA).⁷⁶ The addition of an ACEI to ARB therapy might be beneficial by reducing plasma AII levels, thereby reducing the formation of Ang IV. However, recent studies are conflicting as to whether or not elevations in AII levels, while on ARB therapy, pose a risk since a direct link between plasma Ang IV and PAI-1 levels has not been confirmed.^{58,74,77-79}

Incomplete AII-AT₁ Receptor Blockade

Usual clinical doses of ARBs produce incomplete blockade of AT₁ receptors and the increased AII levels in plasma and extrarenal tissues counteract (to an unknown degree) their effects at the AT₁ receptor. Thus, the rise in plasma AII levels may compete with the ARB at the AT₁ receptor site, thereby reducing its effectiveness. One possible benefit of using an ACEI/ARB combination is to block the reactive increase in plasma AII in response to ARBs. Combination of an ACEI with an ARB prevents the rise in plasma AII levels that occurs with ARB therapy alone.^{14,15}

However, different ARBs display varying degrees of affinity for the AT₁ receptor. Using ARBs with a high affinity for the AT₁ receptor (e.g., the ‘insurmountable’ antagonists, candesartan and irbesartan) compared with those with lower affinity (e.g., the surmountable antagonists, eprosartan and losartan) will determine whether elevated AII levels while on ARB therapy can overcome AT₁ blockade.⁸⁰ As a result, ARBs with high affinity for the AT₁ receptor would not be displaced from the receptor, regardless of the plasma or tissue concentrations of AII. Thus, the addition of an ACEI to decrease plasma AII levels, in order to reduce compensatory responses to AT₁ receptor blockade, may be more important for surmountable ARBs than for insurmountable ARBs.

Inhibition of Tissue ACE Activity

Tissue ACE activity is one component of the tissue RAAS and thus the effectiveness of ACEI therapy is not only dependent on inhibition of circulating ACE activity but also on its ability to antagonize tissue ACE activity. Perhaps tissue ACE inhibition is more important for blocking the long-term actions of the RAAS and for conferring maximal cardio- and renoprotective effects (Table 1).^{81,82} In order effectively to block the actions of the RAAS and to inhibit fully tissue ACE activity, it is necessary to use ACEI at doses higher than normally prescribed.⁹ Some ACEI, due to their lipophilic properties (e.g., ramipril and quinapril), are better able to block tissue ACE activity than others (e.g., enalapril).²⁵ Clinical studies such as MERCATOR, MARCATOR and QUIET, which have examined the potential benefit of ACE inhibitors with high tissue ACE-binding properties have, however, been disappointing. 83-85 Whether or not one ACEI is better able to inhibit tissue ACE activity than another is still inconclusive. The importance of blocking the local RAAS, however, is vital in order to confer maximal target organ protection. It is unknown whether combining ACEI and ARB can block the tissue-RAAS more effectively than monotherapy, but it may be done more easily at lower doses of each drug.

Kinins

Because ACE is identical to kininase II (which inactivates the nonapeptide bradykinin), several studies have reported that potentiation of kinins might be responsible for the additional effects of ACEI (FIG. 3).⁸⁶ Activation of the bradykinin B2 receptor results in the release of NO and prostacyclin, potent endothelial-derived local vasodilator substances.⁸⁷ Interestingly, ARB therapy may also result in higher bradykinin levels, not due to interference in bradykinin metabolism, but secondary to AT₂ receptor stimulation.⁶⁷ Recent studies have reported that bradykinin and nitric oxide levels are increased in response to ARB therapy due to AT₂ receptor stimulation, which can lead to similar physiological effects from kinins as those found following the use of ACEI.^{67,88}

The prime physiological action of kinins is to promote local vasodilation (e.g., improving coronary blood flow) and natriuresis. Kinins have been recently reported to have important short-term effects on BP, but results from previous studies have been conflicting.⁸⁹ However, it has yet to be demonstrated whether kinins exert any important long-term effects on BP control.⁹⁰

Kinins respond to acute and chronic changes in salt and water intake, as do renin and aldosterone. Thus, the antihypertensive effect of kinins follows the activity of the RAAS and kinins are ineffective in lowering BP in low-renin hypertension.^{86,91} Conversely, in sodium-depleted states, kinins are stimulated, thereby serving to dampen or offset the effects of enhanced AII levels and increased activation of the RAAS. In syndromes of disease where vasoactive peptide systems are stimulated and serve to regulate tissue blood flow, kinins may play an important role to antagonize or counterbalance the effects of powerful vasoconstrictor systems, such as the RAAS.

It is well known that ACEI prevents the degradation of bradykinin, but less widely known is the finding that combining an ARB to ACEI therapy does not affect kinin degradation or may actually result in reduced degradation.⁹² Thus, one of the proposed benefits of ACE inhibition, kinin stimulation, is preserved and unaffected by the addition of an ARB and thus may represent an advantage of the ACEI and ARB combination, even over high-dose ARB monotherapy.

Angiotensin Peptide, Ang-[1-7]

The use of ACEI or ARBs results in compensatory stimulation of the RAAS, via inhibition of the negative feedback signal, resulting in greatly elevated Ang I and AII levels. Levels of angiotensin peptides, such as Ang-[1-7], a byproduct of Ang I or AII metabolism, are elevated in patients on ACEI but not ARB monotherapy (FIG. 3).⁹³ Ang-[1-7] is a vasodilator peptide that has actions opposite to those of AII. Under normal conditions, Ang-[1-7] does not play an important role, since it is rapidly degraded by ACE; this may explain why Ang-[1-7] levels are not elevated in patients on ARBs (FIG. 3).⁹³ However, following chronic ACEI therapy, elevation in Ang-[1-7] levels may serve to potentiate the vasodilator actions of bradykinin by helping to stimulate the release of NO, prostaglandins and prostacyclin.¹³ Adding an ACEI to ARB therapy would reduce degradation of Ang-[1-7] and thus potentiate its local vasodilator effect, potentially contributing to the tissue-protective effects of RAAS blockade. It was recently reported that levels of Ang-[1-7] were elevated on combined ARB and ACEI therapy, providing additional vasodilation in rats.⁹⁴

The pharmaceutical compositions disclosed herein are prepared in accordance with standard procedures and are administered at dosages that are selected to reduce, prevent or eliminate the condition (See, e.g., Remington's Pharmaceutical Sciences, Mack Publishing Company, Easton, Pa., and Goodman and Gilman's The Pharmaceutical Basis of Therapeutics, Pergamon Press, New York, N.Y., for a general description of the methods for administering various agents for human therapy).

Pharmaceutically acceptable compositions of the present invention comprise one or more ARBs and one or more ACEI, optionally in association with one or more nontoxic, pharmaceutically acceptable carriers and/or diluents and/or excipients, collectively referred to herein as “carrier” materials, and, if desired, other active ingredients.

The pharmaceutical compositions of the present invention may be administered by any route, preferably in the form of a pharmaceutical composition adapted to such a S route, and would be dependent on the condition being treated. The compounds and compositions may, for example, be administered orally, intravascularly, intramuscularly, subcutaneously, intraperitoneally, or topically. Preferred routes of administration include oral and intravenous administration.

For oral administration, the pharmaceutical compositions disclosed herein may be in the form of, for example, a tablet, capsule, suspension or liquid. The pharmaceutical composition is preferably made in the form of a dosage unit containing a therapeutically effective amount of the active ingredient. Examples of such dosage units are tablets and capsules. For therapeutic purposes, the tablets and capsules can contain, in addition to the active ingredient, conventional carriers such as binding agents, for example, acacia gum, gelatin, polyvinylpyrrolidone, sorbitol, or tragacanth; fillers, for example, calcium phosphate, cellulose, glycine, lactose, maize-starch, mannitol, sorbitol, or sucrose; lubricants, for example, magnesium stearate, polyethylene glycol, silica, or talc; disintegrants, for example potato starch, flavoring or coloring agents, or acceptable wetting agents. Oral liquid preparations generally in the form of aqueous or oily solutions, suspensions, emulsions, syrups or elixirs may contain conventional additives such as suspending agents, emulsifying agents, non-aqueous agents, preservatives, coloring agents and flavoring agents. Examples of additives for liquid preparations include acacia, almond oil, ethyl alcohol, fractionated coconut oil, gelatin, glucose syrup, glycerin, hydrogenated edible fats, lecithin, methyl cellulose, methyl or propyl para-hydroxybenzoate, propylene glycol, sorbitol, or sorbic acid.

The pharmaceutical compositions disclosed herein can be delivered using controlled or sustained release delivery systems (e.g., capsules, bioerodable matrices). Exemplary delayed release delivery systems for drug delivery that would be suitable for administration of the pharmaceutical compositions disclosed herein are described in U.S. Pat. No. 5,990,092 (issued to Walsh); U.S. Pat. No. 5,039,660 (issued to Leonard); U.S. Pat. No. 4,452,775 (issued to Kent); and U.S. Pat. No. 3,854,480 (issued to Zaffaroni).

The pharmaceutical compositions may also be administered via injection. Formulations for parenteral administration may be in the form of aqueous or non-aqueous isotonic sterile injection solutions or suspensions. These solutions or suspensions may be prepared from sterile powders or granules having one or more of the carriers mentioned for use in the formulations for oral administration. The compounds may be dissolved in polyethylene glycol, propylene glycol, ethanol, corn oil, benzyl alcohol, sodium chloride, sterile water, and/or various buffers.

For topical use the compounds of the present invention may also be prepared in suitable forms to be applied to the skin, or mucus membranes of the nose and throat, and may take the form of creams, ointments, liquid sprays or inhalants, lozenges, or throat paints. Such topical formulations further can include chemical compounds such as dimethylsulfoxide (DMSO) to facilitate surface penetration of the active ingredient. Suitable carriers for topical administration include oil-in-water or water-in-oil emulsions using mineral oils, petrolatum and the like, as well as gels such as hydrogel. Alternative topical formulations include shampoo preparations, oral pastes and mouthwash.

Passive transdermal delivery may be useful for delivering some drugs, particularly drugs which are neither charged nor highly hydrophilic. Because passage of hydrophilic molecules across the outermost layer of the skin would be likely to be very low and highly variable, a passive patch would likely be large and need to be worn on a daily basis. Accordingly, U.S. Pat. No. 5,735,810, issued Apr. 7, 1988, to Sage et al. describes an apparatus and method for iontophoretic administration of a compound over several hours, at various dosing intervals. This avoids side effects of oral dosing and the expense, side effects, and discomfort associated with administering an intravenous infusion over several hours.

For rectal administration the compounds of the present invention may be administered in the form of suppositories admixed with conventional carriers such as cocoa butter, wax or other glyceride.

Alternatively, the pharmaceutical compositions of the present invention may be in powder form for reconstitution at the time of delivery.

The dosage regimen for treating an AII-mediated tissue effect, either with high dose ARBs or with the pharmaceutical compositions of this invention, is selected in accordance with a variety of factors, including the type, age, weight, sex and medical condition of the subject, the severity of the AII-mediated tissue effect, the route and frequency of administration, the renal and hepatic function of the subject, and the particular compound or combination of compounds employed. An ordinarily skilled physician or clinician can readily determine and prescribe the effective amount of the pharmaceutical composition required to treat an AII-mediated tissue effect. In general, dosages are determined in accordance with standard practice for optimizing the correct dosage for treating an AII-mediated tissue effect. In certain preferred embodiments the dosage is selected or adjusted independently of an effect on blood pressure.

The dosage regimen can be determined, for example, by following the response to the treatment in terms of aneurysm dilatation, the urinary protein:creatinine ratio, a 24-hour urinary albumin, the severity and frequency of signs and symptoms of CHF, invasive or non-invasive assessment of vascular anatomy (e.g., angiography, echocardiography), and serum or tissue levels of cytokines characteristically induced as part of AII-mediated tissue effect. Harrison's Principles of Internal Medicine, 14th Ed., Fauci AS et al., eds., McGraw-Hill, New York, 1998.

For use as monotherapy, dosages of the ARBs will be any amount greater than an amount corresponding to the currently recommended or approved maximum amount for the treatment or control of hypertension. Typically, dosages of the ARBs will be at least one-and-a-half times, more preferably at least two times, and most preferably at least three times to about twenty times, an amount corresponding to the currently recommended or approved maximum amount for the treatment or control of hypertension. In some embodiments the dosage of ARB will be any amount greater than an amount corresponding to an effective amount for the treatment or control of hypertension in the subject being treated. Typically, dosages of the ARBs will be at least one-and-a-half times, more preferably at least two times, and most preferably at least three times to about twenty times, an amount corresponding to an effective amount for the treatment or control of hypertension in the subject being treated. Any ARB, including but not limited to the agents shown in Table 3, may be used. The amount of ARB will vary with the particular agent selected for use, as evident from Table 3. As an example, dosages of candesartan cilexetil will typically fall within the range of about 48 to 128 mg/day or more. Other agents will have correspondingly higher or lower dosages, reflective of their different usually effective and maximum recommended dosages (Table 3).

In some embodiments a plurality of ARBs may be used as ARB monotherapy. In some embodiments the ARB or ARBs may be used in conjunction with at least one other agent, including aspirin, beta-blocker, aldactone, other compounds that can inhibit atherogenesis or platelet adhesion, diuretics, peripheral adrenergic blockers, central adrenergic stimulants, calcium channel blockers, vasodilators, and other antihypertensive agents excluding ACEI. Agents in these classes are well known in the art and can be found listed and described in, for example, Harrison's Principles of Internal Medicine, 14th Ed., Fauci AS et al., eds., McGraw-Hill, New York, 1998, and the Physician's Desk Reference, 55^{th} Ed., Medical Economics Company, Montvale, N.J., 2001.

For methods calling for treatment with an ARB in combination with an ACEI, in certain embodiments the amount of ARB and the amount of ACEI together is more effective for reducing the AII-mediated tissue effect in the subject than either the amount of ARB alone or the amount of ACEI alone. In some embodiments the amount of ARB and the amount of ACEI together is (1) effective for reducing the AII-mediated tissue effect in the subject and (2) more than is effective for reducing or controlling blood pressure of the subject. In certain embodiments the amount of ARB and the amount of ACEI together is (1) effective for reducing the AII-mediated tissue effect in the subject and (2) more than an amount effective for achieving essentially a same degree of blood pressure reduction or blood pressure control in the subject. In yet another embodiment, in which the subject to be treated does not have hypertension, the amount of ARB and the amount of ACEI together is (1) effective for reducing the AII-mediated tissue effect in the subject and (2) more than an amount that is usually effective for reducing or controlling blood pressure in hypertensive subjects.

The amount of ARB according to this aspect of the invention may be, but need not be, more than an amount corresponding to the currently recommended or approved maximum amount for the treatment or control of hypertension. In certain embodiments the amount of ARB used in combination with ACEI is less than or equal to an amount corresponding to the currently recommended or approved maximum amount for the treatment or control of hypertension. Nevertheless, dosages of the ARB used in combination with ACEI may be at least one-and-a-half times, more preferably at least two times, and most preferably at least three times to about twenty times, an amount corresponding to the currently recommended or approved maximum amount of ARB for the treatment or control of hypertension.

The amount of ACEI according to this aspect of the invention may be, but need not be, more than an amount corresponding to the currently recommended or approved maximum amount for the treatment or control of hypertension. In certain embodiments the amount of ACEI used in combination with ARB is less than or equal to an amount corresponding to the currently recommended or approved maximum amount for the treatment or control of hypertension. Nevertheless, dosages of the ACEI used in combination with ARB may be at least one-and-a-half times, more preferably at least two times, and even more preferably at least three times to about twenty times, an amount corresponding to the currently recommended or approved maximum amount of ACEI for the treatment or control of hypertension.

Any ARB and any ACEI, including but not limited to the agents shown in Table 2 and Table 3, may be used in combination. The particular amounts of ARB and ACEI will vary with the particular agents selected for use, as evident from Table 2 and Table 3.

In some embodiments a plurality of ARBs and/or a plurality of ACEI may be used in combination. In some embodiments the combination of ARB and ACEI may be used in conjunction with at least one other agent, including aspirin, beta-blocker, aldactone, other compounds that can inhibit atherogenesis or platelet adhesion, diuretics, peripheral adrenergic blockers, central adrenergic stimulants, calcium channel blockers, and vasodilators.

The invention in another aspect provides a pharmaceutical composition useful for treating an AII-mediated tissue effect in a subject. The pharmaceutical composition includes an amount of ARB and an amount of ACEI, wherein the amount of ARB and the amount of ACEI together is (1) effective for reducing an AII-mediated tissue effect in a subject and (2) more than an amount that is usually effective for reducing or controlling blood pressure in hypertensive subjects. The pharmaceutical composition may optionally include a pharmaceutically acceptable carrier, as described elsewhere herein. According to this aspect of the invention, the amount of ARB may be, but need not be, more than an amount corresponding to the currently recommended or approved maximum amount for the treatment or control of hypertension. In certain embodiments the amount of ARB used in combination with ACEI is less than or equal to an amount corresponding to the currently recommended or approved maximum amount for the treatment or control of hypertension. Nevertheless, amounts of the ARB used in combination with ACEI may be at least one-and-a-half times, more preferably at least two times, and most preferably at least three times to about twenty times, an amount corresponding to the currently recommended or approved maximum amount of ARB for the treatment or control of hypertension.

The amount of ACEI according to this aspect of the invention may be, but need not be, more than an amount corresponding to the currently recommended or approved maximum amount for the treatment or control of hypertension. In certain embodiments the amount of ACEI used in combination with ARB is less than or equal to an amount corresponding to the currently recommended or approved maximum amount for the treatment or control of hypertension. Nevertheless, dosages of the ACEI used in combination with ARB may be at least one-and-a-half times, more preferably at least two times, and even more preferably at least three times to about twenty times, an amount corresponding to the currently recommended or approved maximum amount of ACEI for the treatment or control of hypertension.

Any ARB and any ACEI, including but not limited to the agents shown in Table 2 and Table 3, may be used in preparing the pharmaceutical composition of the invention. The particular amounts of ARB and ACEI may vary with the particular agents selected for use, as evident from Table 2 and Table 3.

In some embodiments a plurality of ARBs and/or a plurality of ACEI may be employed. In some embodiments the pharmaceutical composition having a combination of ARB and ACEI may further include at least one other agent, including aspirin, a beta-blocker, aldactone, another compound that can inhibit atherogenesis or platelet adhesion, a diuretic, a peripheral adrenergic blocker, a central adrenergic stimulant, a calcium channel blocker, and a vasodilator.

The pharmaceutical compositions of the present invention are preferably formulated for oral administration. Other formulations, including those suitable for parenteral administration, are also embraced by the invention. The pharmaceutical compositions of the present invention may preferably be formulated for once-daily or twice-daily administration.

### EXAMPLES

### Example 1

### Effect of High-Dose AII Receptor Blockade in Reducing Urinary Protein Excretion

The optimal doses of ACEIs and/or ARBs for maximal reduction in urinary protein excretion are not known. Moreover, beneficial effects from ARBs, such as tissue protection owing to a more complete blockade of the RAAS, may be independent of blood pressure-lowering by ARBs. This investigation was designed to evaluate whether increasing the dose of candesartan cilexetil, in subjects already on the FDA's maximum recommended daily doses of 32 mg, would induce a further reduction in 24-hour urinary protein excretion in patients with heavy proteinuria (urinary protein excretion >1.5 g/day; mean 4.4±2 g/day).

Despite aggressive BP control, progression to end-stage renal disease still occurs in patients with diabetic or non-diabetic renal disease. Blockade of the tissue and circulating RAAS has been identified as one of the most important strategies to limit the progression of chronic renal disease. However, the optimal doses of ACEIs and/or ARBs, as well as the optimal therapeutic dosing intervals needed to induce maximal reduction in proteinuria, are not known. Previously, investigators have chosen doses of ACEI and/or ARBs to block the tissue RAAS by measurement of maximal beneficial effects on BP control.

Strategies to enhance blockade of the tissue RAAS may result in greater renoprotective effects, by further reducing intraglomerular hydrostatic pressure and glomerular hypertrophy, and providing additional improvements in glomerular permselectivity. Our preliminary (unpublished) observations demonstrated that using supramaximal doses of ARBs (losartan, valsartan, irbesartan and candesartan) resulted in additional reductions in microalbuminuria in subjects with both diabetic and non-diabetic renal disease. Utilizing ARBs rather than ACEI was chosen owing to evidence that ARBs provide enhanced tissue RAAS blockade by prevention of the ACE escape phenomenon and thus more complete blockade of angiotensin II (AII) produced by both ACE and non-ACE pathways.

Furthermore, the excellent tolerability profile of ARBs compared with ACEI was considered important when using supramaximal doses of these drugs. Therefore, this study investigated whether increasing the dose of candesartan cilexetil beyond the maximally recommended FDA doses (32 mg/d) would further enhance the antiproteinuric effect and thereby preserve renal function in subjects with heavy proteinuria.

Methods.

Ten older patients (67±10 years; 80% male) with heavy proteinuria (>1.5 g/day) were started on 16 or 32 mg of candesartan cilexetil daily. Several patients had renal biopsies performed. Eight patients had nephrotic syndrome, four of whom had diabetic nephropathy, two had focal sclerosis, one had membranous nephropathy, and one had postinfectious nephropathy. The two other patients had nephrosclerosis, one being extremely severe and associated with moderate interstitial disease. Most of the patients were already receiving multiple medications for optimal BP control which included the following: calcium channel blockers (CCBs, 80%), diuretics (70%), beta blockers (40%), alpha blockers (40%), and ACEI (40%). Additional concurrent medications included lipid-lowering agents, anti-ischemic drugs and hypoglycemic agents. No effort was made to standardize any class of medications other than ARBs, and no other controls, such as sodium restriction, were instituted as patients were treated using standard office practice procedures.

After 1-2 months of therapy, the dose of candesartan cilexetil was titrated upwards (at 16-32 mg dose increments) to 96 mg/d, while 24-hour urine samples were obtained to measure protein and creatinine. The candesartan doses were increased independently of the need for BP control. Subsequently, the effect of increased doses of candesartan on 24-hour urinary protein and creatinine excretion, BP, serum creatinine and potassium were serially measured.

Baseline data was utilized either prospectively or from past patient records collected close to the time of the clinical observation period. Furthermore, patients were observed in a normal clinical practice setting and complete data collection (e.g., blood testing) was not necessarily obtained at each office visit. Thus, no statistical analysis was performed but general trends are noted.

Results.

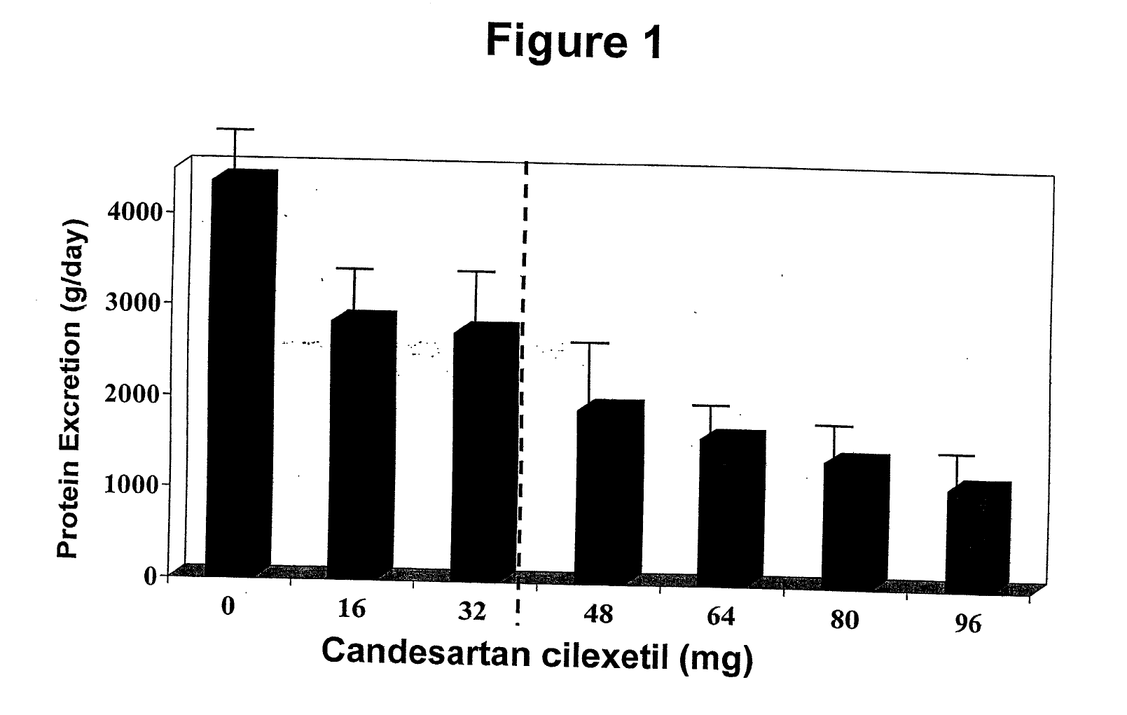

Patients in this clinical study were characterized as normotensive (139±25/80±10 mm Hg systolic/diastolic BP) with reduced renal function (estimated creatinine clearance rate of 69±24 ml/min). The effect of increased doses of candesartan cilexetil demonstrated progressive reductions in 24-hour urinary protein excretion (g/day) beyond the 32 mg/d maximum recommended dose for candesartan cilexetil (FIG. 1).

The 24-hour urinary protein excretion data are presented in Table 4 for individual patients and summarized as the mean±SD. Systolic and diastolic BP appeared similar across all doses of candesartan, although some individuals demonstrated further reductions in BP at doses greater than 32 mg. Additionally, serum creatinine tended to increase by 0.5-0.7 mg/dl at the higher doses of candesartan, although the increase was attributable primarily to two of the ten patients. On the other hand, serum potassium was remarkably consistent, showing similar values from 4.3-4.5 mmol/L throughout the dosing range of candesartan.

Discussion.

Strategies employed to enhance RAAS blockade may be important to preserve existing renal function in patients with progressive renal disease. Utilizing high-dose ARB therapy, above the doses already known to result in no further reductions in BP, may help to further reduce proteinuria and preserve residual renal function. This study demonstrated that, when the dose of candesartan cilexetil was extended beyond the recommended 32 mg/d maximal dose, there were further reductions in urinary protein excretion in all subjects. The additive antiproteinuric effect of high-dose candesartan was largely independent of reductions in BP. In some patients, there were slight increases in serum creatinine when the dose of candesartan cilexetil was increased from 32 to 96 mg. However, candesartan was well-tolerated and appeared safe, since there were no increases in serum potassium values. Thus, using doses of candesartan cilexetil between 32 and 96 mg was safe, well-tolerated, and effective in reducing urinary protein excretion.

Urinary protein excretion may be influenced by many factors such as the etiology of proteinuria, the magnitude of urinary protein excretion at baseline, use of multiple medications (e.g., ACEI, CCBs, diuretics, beta blockers, non-steroidal anti-inflammatory drugs [NSAIDs]), changes in systemic and glomerular pressures, salt intake, drug-protein binding, time-dependent effects and the length of dosing intervals. In this series of clinical observations no attempt was made to control prospectively for any of these factors. Nonetheless, without meaning to be bound by any theory, it is believed that the use of high-dose candesartan for the reduction of proteinuria may have specific advantages over ACEI therapy because of a more complete blockade of the renal RAAS due to the following three factors: 1) heightened intrarenal RAAS activity, 2) the phenomenon of ACE escape and 3) activation of non-ACE pathways for the generation of AII.

TABLE 4
Individual 24-hour urinary protein excretion rates (g/day).

| Candesartan cilexetil | Protein Excretion (g/day) | | | | | | |
|---|---|---|---|---|---|---|---|
| dose(mg/day) | 0 | 16 | 32 | 48 | 64 | 80 | 96 |
| Patient | | | | | | | |
| 1 | 7.7 | — | 6.4 | — | 3.0 | 1.4 | — |
| 2 | 6.5 | 6.4 | 4.6 | 5.8 | 4.7 | 2.4 | — |
| 3 | 2.9 | — | 1.2 | 1.2 | 0.7 | 0.6 | 0.7 |
| 4 | 3.7 | — | 1.1 | 0.9 | 1.7 | 0.9 | 0.6 |
| 5 | 7.5 | 3.2 | 2.0 | — | 1.1 | — | — |
| 6 | 1.7 | 1.1⁺ | 0.4^{§} | 0.2 | 0.4 | 0.5 | 0.2 |
| 7 | 1.7 | 0.5** | 0.8 | 0.2 | 0.2 | 0.3 | — |
| 8 | 4.6 | — | 4.9 | 2.6 | — | — | — |
| 9 | — | — | 3.3 | 1.7 | 1.3 | 1.6 | 1.8 |
| 10 | 3.0 | — | 2.7 | 2.9 | — | 3.4 | 2.3 |
| Mean | 4.4 | 2.8 | 2.7 | 1.9 | 1.6 | 1.4 | 1.1 |
| SD | 2.4 | 2.7 | 2.0 | 1.8 | 1.5 | 1.0 | 0.9 |
| N | 9 | 4 | 10 | 8 | 8 | 8 | 5 |

Values are individual data and averaged (mean) data ± standard deviation (SD).
N = number of patients;
⁺Administered losartan (50 mg);
^{§}Administered irbesartan (300 mg);
**Administered irbesartan (150 mg);
— no data.

Thus, there is evidence to support greater RAAS blockade with ARBs, thereby supporting enhanced benefit of high-dose candesartan therapy over ACE inhibition. In some patients, urinary protein excretion decreased in a dose-dependent fashion above 32 mg of candesartan cilexetil. On the other hand, one individual demonstrated no antiproteinuric response to candesartan cilexetil until 96 mg was administered. Thus, it is the applicant's belief that some subjects may require higher doses of candesartan to achieve regression of proteinuria and optimally preserve renal function. In addition, it is the applicant's belief that increasing the dose of ARBs may require several months for optimal reduction of urinary protein excretion.

### Example 2

### Comparison Between ACEI and ARB Monotherapy for Treatment of Hypertension

To evaluate the effectiveness of combining ACEI and ARBs, a comparison of the antihypertensive responses would help to determine the important mechanisms involved in the lowering of BP for each drug. The antihypertensive mechanism for ACEI has been attributed to reduced formation of AII and for ARBs to a reduced AII binding at the AT₁ receptor. Thus, both of these two classes of drugs block the actions of the RAAS system, but at different sites. If there are any differences in the antihypertensive action of ACEI or ARBs, they must be related to bradykinin-related effects of ACEI or to a more complete blockade of the RAAS with ARBs at the AT₁ receptor. Examining the BP-lowering effects of ACEI in combination with other RAAS blockers such as beta-blockers and renin inhibitors will also provide insight into the value of combining ACEI with ARBs for additive antihypertensive effects.

Even though ACEI and ARBs block the RAAS at different sites, they both prevent plasma AII levels from causing peripheral vasoconstriction and renal Na⁺ retention, the two primary mechanisms involved in the hypertensive response associated with RAAS activation. If the antihypertensive responses to these agents is dependent solely on effective blockade of the circulating RAAS, then the antihypertensive effect of ACEI and ARBs should be similar. Comparative short-term trials between ACEI (e.g., enalapril and lisinopril) and ARBs, such as losartan, valsartan, irbesartan, candesartan, and telmisartan, have all shown equivalent BP reductions. 95-106 This evidence suggests that the bradykinin-related effects of ACE inhibition do not affect long-term BP control. Furthermore, these results suggest that more complete blockade of the RAAS using ARBs, through tissue blockade of both ACE and non-ACE pathways, is not important for BP control. If the bradykinin-related effects of ACEI and more effective tissue blockade of the RAAS with ARBs are physiologically important, they will be related to ensuring more effective protection of end-organ function in clinical syndromes of disease (e.g., diabetic renal disease).

However, a few studies have reported smaller BP reductions with losartan compared with ACEI.¹⁰⁷⁻¹⁰⁹ More recent evidence suggests that losartan may not effectively block the AT₁ receptors throughout the 24-hour dosing period.^{70,110} The less potent antihypertensive effects of losartan compared with enalapril appear to be specific for losartan. Comparative studies of other ARBs against losartan have also consistently reported greater BP lowering effects.¹¹⁰⁻¹¹⁶ Losartan, being a competitive antagonist, displays weak binding properties to the AT₁ receptor and probably accounts for its reduced efficacy when compared with enalapril, rather than any kinin-related effects of ACE inhibition.^{73,117} In summary, BP responses to ARBs and ACEI are generally similar, suggesting similar antihypertensive mechanisms of action. Based on these results, combination therapy with ACEI and ARBs are not expected to result in any additive antihypertensive effects, independent of dose-related effects, and thus, the benefit of combination therapy with ACEI and ARBs would be related to the extent of their long-term beneficial effects on target organ function.

### Example 3

### ACEI in Combination with Renin Inhibitors for Treatment of Hypertension

Renin inhibitors were developed after ACEI and they offer an attractive approach to blocking the RAAS. Although both agents reduce plasma AII levels, renin inhibitors can provide a more effective chronic blockade of AII, unlike ACEI, by preventing the formation of AII through both ACE and non-ACE pathways.^{42,117} Acute human studies have reported a greater renal vasodilator response with renin inhibitors compared with ACEI supporting a more effective tissue RAAS blockade.⁴² However, despite more complete tissue and circulating RAAS blockade with renin inhibitors, no BP lowering differences were reported, in acute and chronic studies, between renin inhibitors and ACEI.^{36,118-121} Based on the different sites of action on the RAAS, adding renin inhibitors to existing ACEI might be expected to provide an additive antihypertensive response. Although some studies reported greater increases in PRA with the combination, there was no additional antihypertensive response.^{122,123} In one study, when renin inhibitors were added to hypertensive guinea pigs already on ACEI, there was a further reduction in BP. However, the reductions in BP were no greater than when one of the drugs was used at its maximum antihypertensive dose.¹²⁴ Thus, combination therapy with ACEI and renin inhibitors will result in more complete RAAS blockade, but not in any additive antihypertensive response.^{125,126} However, the theoretical benefit of more complete RAAS blockade, achieved by combination therapy, is apparent in disease states where the tissue-based RAAS is chronically activated.¹²⁷

### Example 4

### ACEI in Combination with Beta-Blockers for Treatment of Hypertension

Although the precise antihypertensive mechanism of β-blockers is not known, their ability to inhibit sympathetically stimulated renin release is believed to be of chief importance.¹²⁸ A recent study reported that β-blockers are effective in reducing the compensatory rise in renin and Ang I levels in response to ACEI therapy.¹²⁹ The effect of β-blockers on the RAAS is very similar to the actions of renin inhibitors. Like renin inhibitors, -blockers reduce BP in a comparable fashion to ACEI.^{130,131} When combining β-blockers and ACEI in therapy, study results have been mixed, as some studies have reported an additive antihypertensive effect while others reported no additive effect.¹³¹⁻¹⁴⁴ The mechanism for the added hypotensive effect may be unrelated to dual effects on the RAAS, since non-RAAS related antihypertensive effects of β-blockade may be involved. However, the additive antihypertensive effect of combining ACEI and β-blockers is small and is clinically insignificant.^{132,138,141} The primary benefit of combining β-blockers and ACEI is in a more complete inhibition of the RAAS, independent of further BP reduction, in syndromes of disease where the tissue RAAS is chronically stimulated. More complete RAAS blockade, achieved by adding a β-blocker to existing ACEI therapy, may explain some of the positive results recently reported in CHF trials.^{146,147}

### Example 5

### ACEI in Combination with ARBs for Treatment of Hypertension

Previous studies combining another RAAS blocker (e.g., renin inhibitor or β-blocker) with an ACEI have reported little or no additional hypotensive response. However, this was never the primary rationale for using the drugs in combination and neither should it be for ACEI and ARBs. The BP response for the ACEI and ARB combination, based on a review of multiple studies, shows a small additional antihypertensive response.^{13-17,147-162} A variety of clinical studies in hypertension, renal disease and heart failure reported an additive effect on BP when combining the two classes of drugs; however, in most cases, the magnitude of the effect is modest (range 0-6 mm Hg diastolic blood pressure (DBP)) (Table 5).^{16,17,147,153-157,161,162}

TABLE 5
Effect of combination (ARB + ACEI) therapy on blood pressure response
in animal and clinical studies.

| | BP reduction | |
|---|---|---|
| Combination therapy: | ΔSBP/ΔDBP | |
| ACEI and ARBs | (mm Hg) | Reference |
| Hypertensive patients* | 5-7/4-5 | 153-155 |
| Diabetics & renal patients | 2-4/0-6 | 17, 156-157 |
| CHF patients | 2-7/1-3 | 16, 147, 161, 162 |

Changes in systolic (ΔSBP) and diastolic (ΔDBP) blood pressure (mm Hg) from ACEI or ARB monotherapy;
*Comparison of ACEI and ARB combination dose to lower ACEI dose

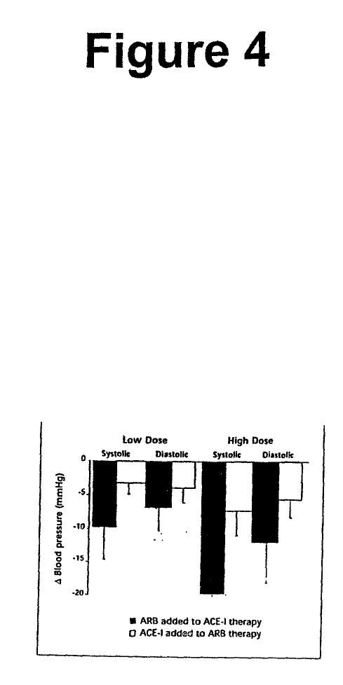

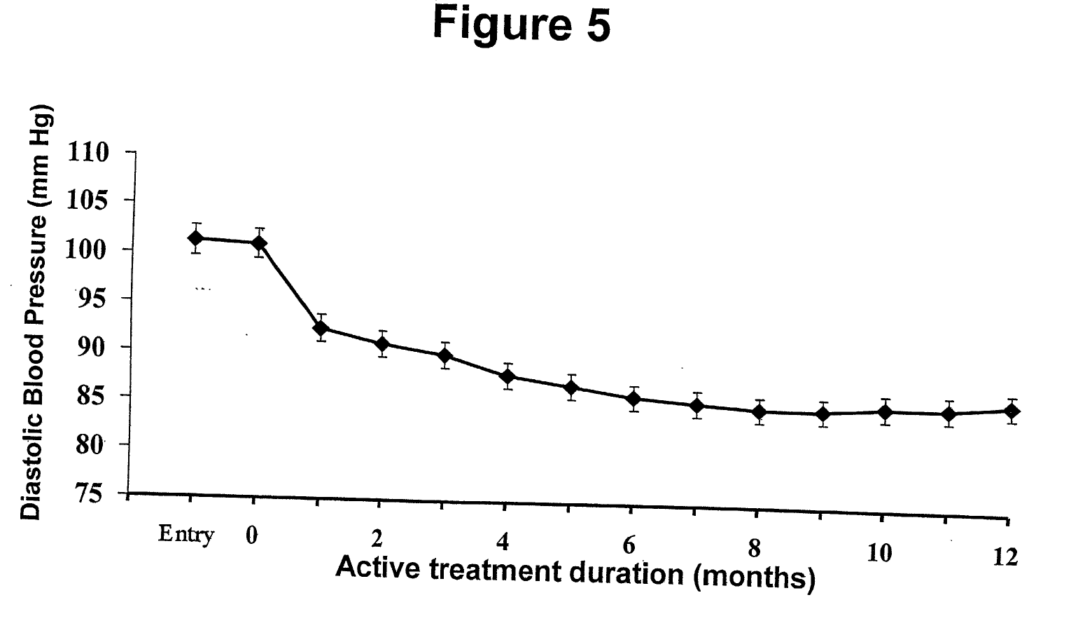

Unfortunately, many of the hypertension studies examining the efficacy of combined ACEI and ARB therapy are difficult to interpret and have the following criticisms: (1) Doses of ACEI used in combination studies are usually not at optimal or FDA-recommended therapeutic levels. Thus, increasing the dose of an ACEI or ARB by itself will result in an additional antihypertensive response, obviating the need of combination therapy. Furthermore, the additive effect of the combination is frequently compared to a relatively lower monotherapy dose, thereby making any conclusions of the benefits of combination therapy difficult to make. Some of the combination studies have reported that the addition of an ARB to ACEI results in a greater antihypertensive response than that obtained by doubling of the ACEI.^{149,150} Since the maximum or optimal antihypertensive effect of monotherapy (ACEI or ARBs) is not known in these studies, it is difficult to attribute a greater antihypertensive response to the combination.²⁶ (2) Using moderate doses, the order of treatment appears to determine the magnitude of the antihypertensive response to the combination. In one four-week study of 30 hypertensives, the antihypertensive response was enhanced when the ARB (losartan, 25-50 mg/d) was added to existing ACEI therapy (ramipril, 2.5-5 mg/d) (FIG. 4).¹⁵¹ Conversely, when the ACEI was added to the existing ARB therapy, the additive antihypertensive response was small and not significant. It is not known if the same results would be observed in the long term, but it warrants further consideration since all studies to date examine the additive effects of an ARB to existing ACEI therapy. (3) In some studies, the addition of ARBs to ACEI therapy does not allow enough time for the antihypertensive agents to exert their maximal effects and reach a steady state. For example, the maximum antihypertensive response reported in clinical trials for ACEI is approximately two weeks, while for ARBs it is four weeks. Interestingly, a recent study reported continuous reductions in BP up to eight months following the initiation of ARB therapy, suggesting a secondary time-dependent antihypertensive response that develops after months of therapy (FIG. 5).¹⁶³ Thus, in acute and short-term studies, it is difficult to separate out the BP-related effects of the combination from the time-dependent BP effects associated with monotherapy. (4) Reductions in dietary sodium consumption and/or diuretic usage will also increase the magnitude of the antihypertensive response to the combination, resulting in larger BP changes than normally observed in the clinical practice setting.

Even if combined therapy of an ACEI and ARB only results in a modest additional antihypertensive response, its use may be more efficient since the same blood pressure reduction can be achieved by combining the ACEI or ARB dose rather than titrating the individual ACEI dose to maximum.¹⁵⁵ However, it is believed by the applicant that there may be an additional advantage in an enhanced ability of the combination to antagonize the circulating and tissue-based system RAAS. Combination studies have reported a greater effect on PRA despite no additional antihypertensive response when ARBs are combined with ACEI.^{13,149} The benefit of more effective RAAS blockade should lead to enhanced tissue-protective effects which can, indirectly, influence long-term BP control. Thus, combining ACEI and ARBs may not lead to additive antihypertensive effects, in response to initial therapy, but is targeted to more effective RAAS blockade, resulting in long-term beneficial tissue-protective effects (e.g., reduced vascular smooth muscle hypertrophy).

### Example 6

### ACEI in Combination with ARBs for Treatment of Chronic or Congestive Heart Failure

During the past several years, the administration of ACEI therapy has become the current standard of care in CHF. Studies with ACEI have reported both hemodynamic and non-hemodynamic benefits in patients with CHF. Improvements in left ventricular (LV) mass, LV ejection fraction, and neurohumoral activity, coupled with benefits in mortality and morbidity outcomes, are well accepted in CHF studies following ACEI therapy. However, despite the well-known benefits of ACEI in CHF, the mortality in CHF remains unacceptably high.^{51,164}

Long-term therapy with ACEI may result in incomplete suppression of the RAAS and may contribute to the progressive worsening in cardiac function in patients with CHF.^{28,165} The following factors may account for the incomplete suppression of the RAAS in CHF: (1) Following an initial reduction in plasma AII levels in patients on ACEI therapy, there is a gradual increase in the plasma concentrations of AII back towards baseline levels (ACE escape).¹⁶⁶ (2) The optimal dose of ACEI in subjects with CHF is not known, despite data from a recent clinical trial (i.e., ATLAS study) which suggested that the optimal dose needs to be higher than that commonly used by practicing physicians.^{32,34} Even using standard doses of ACEI in clinical practice results in incomplete blockade of the RAAS.²⁸ One of the current problems in the management of CHF patients is not so much utilization of ACEI as the underdosing of ACEI therapy.¹⁶⁷ It is recommended, in CHF, that the ACEI be titrated to the highest dose tolerable for optimal cardiovascular protection.²⁸ (3) Recent trials in CHF reported additional benefits derived following the addition of an aldosterone antagonist (e.g., RALES study) or β-blocker (e.g., MERIT-HF study) to existing ACEI therapy. These studies suggest that more complete blockade of the RAAS in CHF may contribute to the mortality benefit and is the primary factor justifying the use of ARBs with ACEI in CHF.¹⁴⁶ Animal and clinical studies examining the combination of ACEI and ARBs have demonstrated hemodynamic improvements, neurohumoral reductions, and beneficial tissue effects on cardiac volumes and left ventricular hypertrophy (Table 6). Two recently completed studies, involving patients on combined ACEI and ARB therapy, reported that the combination group had greater effects on left ventricular volumes and mass, independent of BP changes, compared with ACEI or ARB monotherapy.^{147,160} In both studies, the beneficial effect on cardiac tissue remodeling was associated with a more complete blockade of the RAAS.

These initial beneficial results with the combination group in CHF were all associated with reduced RAAS activation, which may be the most important reason justifying the combined use of ACEI and ARBs in CHF. However, the therapeutic benefit of combined therapy has yet to be proved and is currently being evaluated in large prospective randomized clinical trials in CHF and post-myocardial infarction patients (e.g., CHARM, Val-HeFt; VALIANT) (Table 7).^{69,92,179-181} It is the applicant's belief that the results from these trials will demonstrate that combining ACEI and ARBs for use in CHF provides additional cardiac and vascular protective benefits, independent of BP reduction.

TABLE 6
Potential advantages of combination therapy (ACE + ARB)
in chronic heart failure
Outcome

| | |
|---|---|
| Basic/clinical studies | Enhanced blockade of the RAAS |
| | (e.g., increase in PRA; |
| | reduced plasma All levels)^{16,159,160,168,169} |
| | Reduced neurohormonal activation |
| | (e.g., reduced plasma |
| | norepinephrine and aldosterone levels)^{16,169,170} |
| | Improved CHF functional class¹⁶⁵ |
| | Improved exercise tolerance and increase in peak |
| | VO₂^{161,165,166,169} |
| | Reduced total peripheral resistance and |
| | improved regional |
| | blood flow distribution¹⁷¹⁻¹⁷³ |
| | Improved LV function (e.g., increased |
| | cardiac output and |
| | LVEF)^{147,160,166,170,173-176} |
| | Reduced ventricular volumes (e.g., |
| | ESV, EDV)^{147,160,161} |
| | Additive reductions in LV mass^{177,178} |

RAAS = renin-angiotensin-aldosterone system;
PRA = plasma renin activity;
AII = angiotensin II;
CHF = chronic heart failure;
VO₂ = oxygen uptake;
LV = left ventricle;
LVEF = left ventricular ejection fraction;
ESV = end systolic volume;
EDV = end diastolic volume

TABLE 7
Clinical trials examining the combined use of ACEI and ARBs^{69,92,157,179-181}

| Study name | Doses | Patient type | Efficacy variables | Completion |
|---|---|---|---|---|
| CALM | | | | |
| effect of candesartan | Candesartan cilexetil | 180 type II diabetic | Renal protection study: | 2000 |
| cilexetil and lisinopril on | (16 mg q.d.) and | hypertensive patients | microalbuminuria, | |
| microalbuminuria¹⁵⁷ | lisinopril (20 mg q.d.) | with microalbuminuria | tolerability and BP | |
| VALIANT | | | | |
| valsartan in acute | Valsartan (80 mg b.i.d.) | 14,500 post-MI patients | Post-MI study: morbidity | 2003 |
| myocardial infarction¹⁷⁹ | and captopril | for 3-5 years. | and mortality | |
| | (50 mg t.i.d.) | (endpoint = 2700 deaths) | | |
| Val-HeFT | | | | |
| valsartan | Valsartan (160 mg b.i.d.) | 5200 CHF patients | CHF study: morbidity and | 2000 |
| heart failure tria¹⁶⁹ | added to existing | (class II-IV) for 2-4 years. | mortality, disease progression, | |
| | ACEI therapy | (endpoint = 906 deaths) | cardiac structure and function | |
| | | | and quality of life | |
| CHARM | | | | |
| Candesartan heart | Candesartan | 6500 class II-IV CHF | CHF study: morbidity | 2002 |
| failure assessment of | (16 mg q.d.) | patients for 3-5 years | and mortality, disease | |
| reduction in mortality | and enalapril | | progression, cardiac structure | |
| and morbidity¹⁸⁰ | (10 mg b.i.d.) | | and function and | |
| | | | quality of life | |
| RAAS | | | | |
| Randomized | Losartan (50 mg q.d.) | 120 CHF patients with | CHF study: safety, | 2000 |
| angiotensin receptor | and enalapril | moderate to severe | quality of life, | |
| antagonist - angiotensin- | (10 mg b.i.d.) | LV dysfunction | neurohormonal activation, | |
| converting enzyme | | randomized to 1 or the 3 | exercise performance | |
| inhibitor study^{92,181} | | groups and followed for | | |
| | | 6 weeks | | |

BP = blood pressure;
CHF = chronic heart failure;
MI = myocardial infarction;
LV = left ventricle;
q.d. = dosing once a day;
b.i.d. = dosing twice a day;
t.i.d. = dosing three times a day

### Example 7

### ACEI in Combination with ARBs for Treatment of Chronic Renal Disease

The beneficial action of ACEI, in reducing the progression of chronic renal disease, has been primarily through its direct antagonism of the RAAS by reducing the formation of AII.³³ Following the use of ACEI in renal disease, the beneficial effects of RAAS inhibition have been attributed to a combination of both hemodynamic and non-hemodynamic mechanisms.^{182,183} The non-hemodynamic effects of RAAS blockade have previously demonstrated that ACEI have greater renoprotective effects than all other classes of antihypertensive medications, with the possible exception of the ARB class which is under current investigation.¹⁸² During ACEI therapy, blockade of the renal RAAS helps to ameliorate urinary protein excretion, elevated glomerular capillary pressure, and mesangial cell hypertrophy.¹⁸⁴

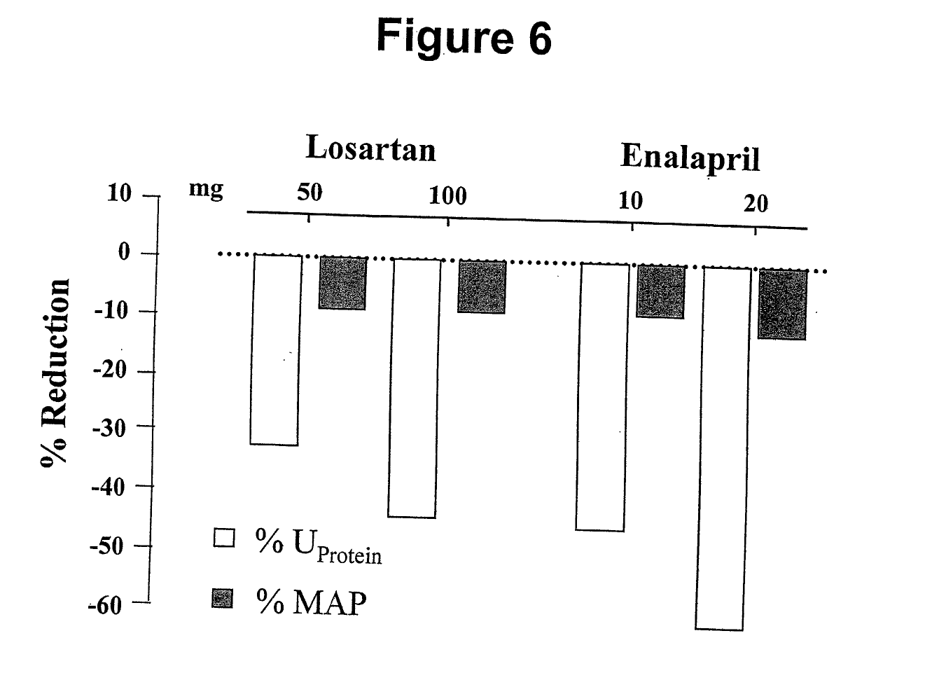

However, ACEI must be used with caution in some syndromes of chronic renal disease. Although ACEI are indicated for use in hypertension, CHF, and diabetic renal disease, the optimal renoprotective dose of ACEI in long-term therapy is not known. The renoprotective effects of RAAS blockers (i.e., using ACEI or ARB) are dose-dependent, with maximum benefit occurring at higher doses at which no additional BP changes have been demonstrated (FIG. 6).¹⁸⁵ It has been recently suggested that improving RAAS blockade by using maximal ACEI doses should yield the greatest reduction in proteinuria.¹⁸⁴ Clinical trials with ACEI have not been designed to evaluate the dose-response curve for renoprotection. Additional renoprotective effects have been reported when the standard dose of ACEI has been increased, which suggests that the doses of ACEI commonly used in clinical practice may be suboptimal in renal disease.¹⁸⁴

A review of the literature reveals that ARBs possess no advantage over ACEI in the reduction of proteinuria and prevention of the progression of renal disease, as evaluated in a variety of animal models such as the spontaneously hypertensive rat, streptozotocin-induced diabetic rat, reduced renal mass, and two-kidney, one-clip hypertension model.^{186,187} Similarly, animal studies examining the combined use of ARBs and ACEI have not demonstrated any additional renoprotective effects over ACEI monotherapy.^{188,189} Using combination therapy, Ots et al. reported no additional renoprotective benefit in reducing urinary protein excretion, or differences in BP response to ACEI and ARB monotherapy.¹⁸⁹ The renoprotective effects of anti-hypertensive animal models correlate very highly with the magnitude of the reduction in BP.¹⁸⁷ Thus, the difficulty in interpreting results from the animal studies to examine the potential benefit of combination therapy may be due to important species differences. In humans, non-ACE pathways (e.g., chymase) have a functional role in the local generation of AII, thereby affecting tissue/organ function. In animal studies, it appears, that BP control and renoprotection are directly correlated, regardless of which antihypertensive agent is used.¹⁹⁰ It is in man, when BP is aggressively controlled, where this relationship does not hold, as studies have reported important non-hemodynamic effects of RAAS blockade beyond systemic BP lowering on renoprotection.^{184,191}

Since AII can be partly generated in human cardiac, vascular and renal tissue by non-ACE pathways, animal studies cannot be used to draw conclusions in human disease models.⁴³ Studies by Hollenberg et al.⁴² have reported that the acute renal vasodilator response to ACEI and ARB therapy in humans correlates with PRA and serves as an acute marker of intrarenal (i.e., tissue) RAAS activity. In these studies, the renal vasodilator response, following administration of ARBs, is enhanced over ACE inhibition, supporting the functional significance of non-ACE pathways in humans and the importance of studying species differences.⁴⁴

However, so far, in the limited number of clinical studies comparing ARBs with ACEI, no differences have been shown between these two groups in terms of renoprotection.¹⁹² In two studies comparing the antiproteinuric effects of losartan with enalapril, both drugs resulted in similar effects on renal hemodynamics and reductions in BP and proteinuria. The additional reductions in urinary protein excretion after doubling the dose in both studies were primarily independent of BP-lowering or hemodynamic effects (FIG. 6).^{33,185}

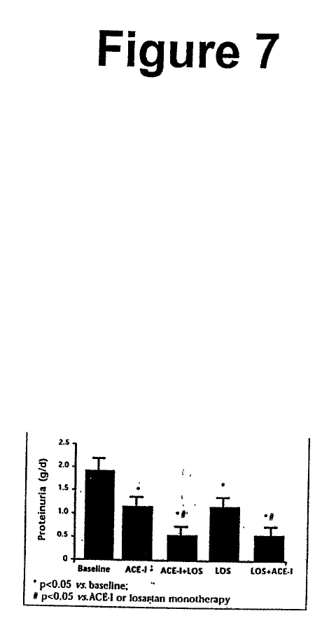

Clinical studies that have combined ACEI and ARBs have reported more complete inhibition of the RAAS, resulting in enhanced renal vasodilation and additional reductions in proteinuria (Table 8).^{10,17,58,156,193-196} In one study, when losartan (50 mg/day) was added to a moderate dose of an ACEI in normotensive patients with IgA nephropathy, proteinuria was more profoundly reduced than with either agent alone (FIG. 7).¹⁹⁴ Urinary protein excretion was reduced by 69% on combined ACEI and ARB therapy compared with either drug as monotherapy (−39% on ACEI; −27% on ARB). The additional effect on urinary protein excretion was not dependent on changes in systemic BP or glomerular filtration rate (GFR), but was secondary to the improvements of hemodynamics and/or membrane permeability at the glomerular level.¹⁹⁴ A recently completed multicenter trial, the CALM study, reported that the combination of lisinopril (20 mg/d) and candesartan (16 mg/d) was more effective than either lisinopril or candesartan alone.²⁰⁰ (See Table 7).

TABLE 8
Potential benefits of combination therapy (ACEI + ARB) in renal disease
patients^{10,17,58,156,193-196}
Outcome

| | |
|---|---|
| Clinical studies | Enhanced blockade of the RAAS (e.g., |
| | increase in PRA)^{20,191} |
| | No change or slight reduction in plasma |
| | aldosterone levels²⁰ |
| | Increased RPF¹⁹¹ |
| | Preserved or no ΔGFR^{20,191} |
| | Reductions in proteinuria^{157,195} |
| | Greater ability to suppress cytokine expression |
| | (e.g., TGF-β₁)^{13,59,193} |

RAAS = renin angiotensin-aldosterone system;
PRA = plasma renin activity;
RPF = renal plasma flow;
GFR = glomerular filtration rate;
TGF-β₁ = transforming growth factor beta-1

### Example 8

### Possible Side Effects

Possible adverse effects associated with the use of ARBs in combination with ACEI are presented in Table 9.^{12,16-17,147,153,155,156,159-162,165,193} Studies have reported the tolerability of the combination to be similar to monotherapy with ACE inhibition. The incidence of cough, the most troublesome and common reaction to ACEI therapy, is also expected to be similar in the combination group.¹⁵³ It has been suggested that renal-impaired patients, who are sensitive to RAAS blockade, particularly with high doses of ACEI, may fare better by using lower doses of ACEI and an ARB to preserve GFR, rather than using a high dose of either drug.^{12,197} In addition, this should lead to less hyperkalemia.¹⁹⁸ A slight increase in serum creatinine (15 μmol/L) was recently reported in a study of renal-impaired patients on combined ARB and ACEI therapy, but the response was not thought serious, since this is a typical effect of ACE inhibition in patients with a reduced GFR. Thus, the use of combination ACEI and ARB therapy in renal and diabetic patients should follow the same protocol as when one initiates ACEI therapy. If an ARB is to be added to existing ACEI therapy, a low dose should be initiated and titrated upwards thereafter based on need.

TABLE 9
Possible adverse effects associated with the use of ARBs in
combination with ACEI^{12,16,17,147,153,155,156,159-162,165,193}

| Proposed adverse effect | Incidence/outcome |
|---|---|
| Hypotension, dizziness | Hypertensive patients - mild or |
| | not observed^{153,155} |
| | Diabetic patients - transient episode |
| | in 2/7 patients¹⁷ |
| | CHF and post-MI patients - not observed^{159,160} |
| Tachycardia | Not observed^{155,161} |
| Reduced GFR | Hypertensive patients - not observed¹⁹³ |
| (acute renal failure) | Renal-impaired patients - slight increase |
| | in serum creatinine levels |
| | (0.15 μmol/L), no acute renal |
| | failure or progressive renal failure |
| | observed^{156,162} |
| Cough | Incidence of coughing similar to |
| | ACEI monotherapy¹⁵³ |
| Angioneurotic edema | Observed in one combined therapy study, |
| | thus potential still exists; |
| | follow same precaution as ACEI¹⁶² |
| Hyperkalemia | Plasma aldosterone levels not affected |
| in | in hypertensive and diabetic |
| | patients^{13,17} |
| | Plasma aldosterone levels slightly |
| | reduced in CHF patients^{16,147,160} |
| | No significant changes in plasma potassium |
| | or reports of hyperkalemia |
| | hypertensive or diabetic patients¹³ |
| | Slight increase (+0.11) in |
| | plasma potassium concentration in CHF |
| | patients^{149,162} |
| | Plasma potassium levels slightly increased |
| | in renal-impaired patients but |
| | incidence of hyperkalaemia was low¹⁵⁶ |
| Decompensation in heart | Aggravated CHF symptoms and fluid |
| faulure patients | overload incidence low, similar |
| | to placebo^{161,162,165} |
| Anaphylactoid/sensitivity | Not observed |
| reactions | |
| Fetal/neonatal morbidity | Same contraindications as ACEI and ARBs |
| and mortality | |

CHF = chronic heart failure;
GFR = glomerular filtration rate;
MI = myocardial infarction

In a recently completed large CHF study (n=768 patients), discontinuation rates did not differ between the combination and the ACEI therapy group.¹⁴⁷ Therefore, side effects should not be different with combination therapy when compared with low-dose ACEI monotherapy. To date, there are no data to suggest that side effects using the combination are any greater than that expected with the agents alone as monotherapy. Perhaps, if the dose of the ACEI needs to be increased and there is a concern, lower doses of both drugs can be used for greater RAAS blockade than ACEI alone.

### Example 9

### Use of High-Dose ARB, or ACEI in Combination with ARB, for Treatment of Aneurysms

Applicant believes that in order to preserve the health of blood vessels and prevent the progression of aortic aneurysms, the use of high dose ARBs will allow penetration into the vascular diseased tissue, and the AT₁ receptor, which in some disease states is compartmentalized and internalized, can be found by using high doses of an ARB in doses much greater than that required for BP control. As described herein, it has been shown that 96 mg/d of candesartan is well tolerated, metabolically neutral, and reduces heavy proteinuria or microalbuminuria to zero or nearly zero. Applicant believes the same will hold true when using most any ARB in the market, e.g., ATACAND®, AVAPRO®, BENICARTM, COZAAR®, DIOVAN®, MICARDIS®, and TEVETEN® (see Table 3).

### Example 10

### Reduction in Aortic Root Size with ARB

Aortic root size is believed to increase with age. The role of blood pressure on aortic root size, independent of age, is not settled. The natural trend of aortic root sizes to increase over time has been reported to follow the following regression equations:

AORoot (males)=27.83+0.0612×age+0.0234×MAP

AORoot (females)=22.65+0.0755×age+0.0224×MAP

where AORoot is aortic root size in millimeters (mm) as measured using two-dimensional echocardiographically guided M-mode echocardiography with a leading-edge-to-leading-edge measurement of the maximal distance between the anterior aortic root wall and the posterior aortic root wall at end diastole, age is measured in years, and MAP refers to mean arterial pressure in mm Hg. Vasan RS et al. (1995) Circulation 91:734-40.

One subject receiving ARB for microalbuminuria was incidentally noted to have a large reduction in aortic root dilatation as the dose of the ARB ATACAND® was increased to supramaximal doses (greater than that approved by the FDA for the treatment of hypertension). Since regression of the aortic root dilatation on echocardiogram was so unusual, a study was undertaken to evaluate other individuals with aortic root dilatation to determine whether aortic root aneurysms or dilatation could be reduced using ARBs.

Thirty-two patients had an echocardiogram performed and were found to have dilated aortic roots for a variety of medical diseases other than coronary artery disease (CAD), Marfan syndrome and Ehlers-Danlos syndrome type IV. In order to evaluate one group of homogeneous subjects, the following were inclusion criteria: (1) same modality of testing to determine the thickness of the aortic root; (2) same instrument; (3) same interpreter of echocardiogram; (4) subjects needed to be on medication (antihypertensive including ARB or ARB if normotensive); (5) at least 9 months of ARB therapy; and (6) the disease was strictly aneurysm, not CAD or CHF. Applying the above inclusion criteria, a group of 15 subjects was selected for analysis. The group included 13 men and 2 women, aged 66.4±13.3 years and weighing 188.8±29.6 pounds.

Aortic root sizes were measured using M-mode echocardiography with a leading-edge-to-leading-edge measurement of the maximal distance between the anterior aortic root wall and the posterior aortic root wall, measured at a level immediately above the cusps at end diastole. The standard deviation for intra-observer error was ±2 mm. Interval changes in aortic root size were measured as initial value—most recent value, where a positive resulting value indicated a corresponding decrease in aortic root size over the interval.

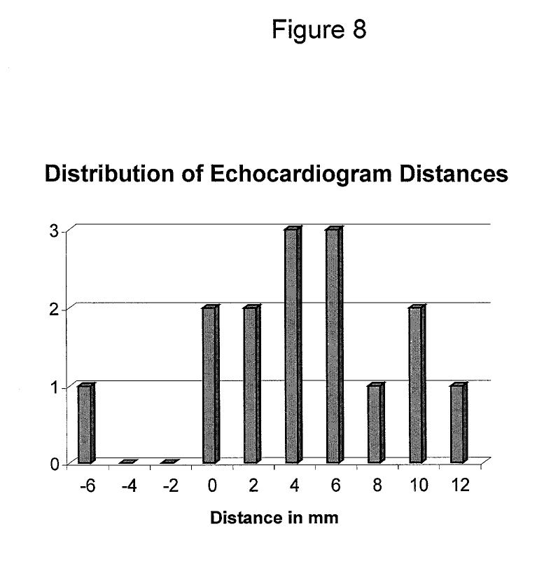

Results are depicted in FIG. 8, which shows that all but one subject had a decrease or no change in aortic root size over the interval, and ten subjects had decreases of more than 2 mm. One subject with an interval change of 12 mm was normotensive and received ATACAND® 64 mg/d. One subject with an interval change of 8 mm was hypertensive and received ATACAND® 128 mg/d.

### REFERENCES

1. Pitt B. ACE inhibition in heart failure: prospects and limitations. Cardiovasc Drugs Ther 1997; 11: 285-90.

2. Remuzzi G. Editorial. Renoprotective effect of ACE-inhibitors: dissecting the molecular clues and expanding the blood pressure goal. Am J Kidney Dis 1999; 34(5): 951-3.

3. Biollaz J, Brunner HR, Gavras I, Waeber B, Gravas H. Antihypertensive therapy with MK 421: angiotensin II-renin relationships to evaluate efficacy of converting enzyme blockade. J Cardiovasc Pharmacol 1982; 4(6): 966-72.

4. Mooser V, Nussberger J, Juillerat L et al. Reactive hyper-reninemia is a major determinant of plasma angiotensin II during ACE inhibition. J Cardiovasc Pharmacol 1990; 15(2): 276-82.

5. Nussberger J, Juillerat L, Perret F, Waeber B, Bellet M, Brunner J, Menard J. Need for plasma angiotensin measurements to investigate converting-enzyme inhibition in humans. Am Heart J 1989; 117(3): 717-22.

6. Dzau V J. Short- and long-term determinants of cardiovascular function and therapy: contributions of circulating and tissue renin-angiotensin systems. J Cardiovasc Pharmacol 1989; 14(Suppl 4): S1-5.

7. Johnston C I. Renin-angiotensin system: a dual tissue and hormonal system for cardiovascular control. J Hypertens 1992; 10(Suppl 7): S13-26.

8. Dzau V J, Re R.Tissue angiotensin system in cardiovascular medicine: a paradigm shift? Circulation 1994; 89: 493-98.

9. Rakugi H, Wang D S, Dzau V J, Pratt R E. Potential importance of tissue angiotensin-converting enzyme inhibition in preventing neointima formation. Circulation 1994; 90(1): 449-55.

10. Peters H, Ritz E. Dosing angiotensin II blockers—beyond blood pressure. Nephrol Dial Transplant 1999; 14: 2568-70.

11. Ruilope L M. Is it wise to combine an ACE inhibitor and an angiotensin receptor antagonist? Nephrol Dial Transplant 1999; 14(12): 2855-6.

12. Azizi M, Menard J. Combined blockade of the renin-angiotensin system. Arch Mal Coeur Vaiss 1999; 92(6): 735-9.

13. Webb R L, Navarrete A E, Davis S, deGasparo M. Synergistic effects of combined converting enzyme inhibition and angiotensin II antagonism on blood pressure in conscious telemetered spontaneously hypertensive rats. J Hypertens 1998; 16(6): 843-52.

14. Menard J, Campbell D J, Azizi M, Gonzales M F. Synergistic effects ACE inhibition and AII antagonism on blood pressure cardiac weight and renin in spontaneously hypertensive rats. Circulation 1997; 96(9): 3072-8.

15. Azizi M, Chatellier G, Guyene T T, Murieta-Geoffroy D, Menard J. Additive effects of combined angiotensin on blood pressure and renin release in sodium deplete normotensives. Circulation 1995; 92(4): 825-34.

16. Baruch L, Anand I, Cohen I S, Ziesche S, Judd D, Cohn J N. Augmented short- and long-term hemodynamic and hormonal effects of an angiotensin receptor blocker added to angiotensin-converting enzyme inhibitor therapy in patients with heart failure. Circulation 1999; 99(20): 2658-64.

17. Hebert L A, Falkenhain M E, Nahman N S, Cosio F G, Dorisio T M. Combination ACE inhibitor and angiotensin II receptor antagonist therapy in diabetic nephropathy. Am J Nephrol 1999; 19(1): 1-6.

18. Juillerat L, Nussberger J, Menard J et al. Determinants of angiotensin II generation during converting enzyme inhibition. Hypertension 1990; 16(5): 564-72.

19. MacFadyen R J, Lee A F, Morton J J, Pringle S D, Struthers A D. How often are angiotensin II and aldosterone concentrations raised during chronic ACE inhibitor treatment in cardiac failure? Heart 1999; 82: 57-61.

20. Roig E, Perez-Villa F, Morales M, Jimenez W, Orus J, Heras M, Sanz G. Clinical implications of increased plasma angiotensin II despite ACE inhibitor therapy in patients with congestive heart failure. Eur Heart J 2000; 21(1): 53-7.

21. Van den Meiracker A H, Manin-T-Veld A J et al. The pharmacokinetic and pharmacodynamic interactions of ramipril with propranolol. Eur J Clin Pharmacol 1993; 45(3): 255-60.

22. Nussberger J, Brunner D B, Waeber B, Brunner H R. Plasma angiotensins under sustained converting enzyme inhibition with enalapril in normal humans. J Hypertens 1985; 3(Suppl 3): S269-70.

23. Nussberger J, Waeber G, Waeber B, Bidiville J, and Brunner H R. Plasma angiotensin-(1-8) octapeptide measurement to assess acute angiotensin-converting enzyme inhibition with captopril administered parenterally to normal subjects. J Cardiovasc Pharmacol 1988; 11(6):716-21.

24. Campbell D J. Endogenous angiotensin II levels and the mechanism of action of angiotensin-converting enzyme inhibitors and angiotensin receptor type 1 antagonists. Clin Exp Pharmacol Physiol 1996; (Suppl 3): S125-31.

25. Pinto Y M, van Geel P P, Alkfaji H, van Veldhuisen D J, van Gilst W H. Dosing of ACE inhibitors in left ventricular dysfunction: does current clinical dosing provide optimal benefit? J Cardiovasc Pharmacol 1999; 34(Suppl 1): S13-7.

26. Sander G E, McKinnie J J, Greenberg S S, Giles T D. Angiotensin-converting enzyme inhibitors and angiotensin II receptor antagonists in the treatment of heart failure caused by left ventricular systolic dysfunction. Prog Cardiovasc Dis 1999; 41(4): 265-300.

27. Arzilli F, Magagna A, Giovannefti R, Nuccorini A, Salvetti A. Acute dose-response curve of enalapril in renovascular hypertensives. Am J Hypertens 1988; 1(3): S75-8.

28. Jorde U P, Ennezat P V, Lisker J et al. Maximally recommended doses of angiotensin-converting enzyme (ACE) inhibitors do not completely prevent ACE-mediated formation of angiotensin II in chronic heart failure. Circulation 2000; 101(8): 844-6.

29. Cirillo V J, Gomez H J, Salonen J et al. Lisinopril: dose-peak effect relationship in essential hypertension. Br J Clin Pharmacol 1988; 25(5): 533-8.

30. Schnaper H W. Dose-response relationship of ramipril in patients with mild-to-moderate hypertension. J Cardiovasc Pharmacol 1991; 18(Suppl 2): S128-30.

31. Frishman W H: The safety and efficacy of quinapril in the treatment of mild to moderate essential hypertension. Clin Cardiol 1990; 6(Suppl 7):VII19-VII25.

32. Brunner-La Rocca H P, Weilemnann D, Kiowski W, Maly F E, Candinas R, Follath F. Within-patient comparison of effects of different dosages of enalapril on functional capacity and neurohormone levels in patients with chronic heart failure. Am Heart J 1999; 138(4): 654-62.

33. Gansevoort R T, deZeeuw D, dejong P E. Is the antiproteinuric effect of ACE inhibition mediated by interference in the renin-angiotensin system? Kidney Int 1994; 45(3): 861-7.

34. Packer M, Poole-Wilson P A, Armstrong P W, Cleland J G F, Horowitz J D, Massie B M, Ryden L, Thygesen K, Uretsky B F for the ATLAS Study Group. Comparative effects of low and high doses of the angiotensin-converting enzyme inhibitor lisinopril on morbidity and mortality in chronic heart failure. Circulation 1999; 100: 2312-8.

35. Palla R, Panichi V, Finato V et al. Effect of increasing doses of lisinopril on proteinuria of normotensive patients with IgA nephropathy and normal renal function. Int J Clin Pharmacol Res 1994; 14(1): 35-43.

36. Blaine E H. Characteristics of the blood pressure lowering action of renin inhibition and comparison with angiotensin-converting enzyme inhibition. J Hypertens 1989; 7(Suppl 2): S47-9.

37. Inglessis N, Nussberger J, Hagmann M et al. Comparison of the angiotensin II antagonist UP2696 with the angiotensin-converting enzyme inhibitor enalapril in normotensive volunteers challenged with angiotensin I. J Cardiovasc Pharmacol 1995; 25(6): 986-93.

38. Nussberger J, Waeber B, Brunner H R. Plasma angiotensin II and the antihypertensive action of angiotensin-converting enzyme inhibition. Am J Hypertens 1989; 2(4): 286-93.

39. Salgado M C, Caldo H, Pinheiro M A. Discrepancy between plasma angiotensin-converting enzyme activity and in vivo extent of angiotensin I conversion in hypertensive rats. Braz J Med Biol Res 1991; 24(3): 311-8.

40. Asmar R G, Benetos A, Dame B M, Pauly N C, Safar M E. Converting enzyme inhibition: dissociation between antihypertensive and arterial effects. J Hum Hypertens 1992; 6(5):381-385.

41. Borland J A A, Chester A H, Morrison K A, Yacoub M H. Alternative pathways of angiotensin II production in the human saphenous vein. Br J Pharmacol 1998; 125(3): 423-8.

42. Hollenberg N K, Fisher N D L, Price D A. Pathways for angiotensin II generation in intact human tissue. Evidence from comparative pharmacological interruption of the renin system. Hypertension 1998; 32: 387-92.

43. Okunishi H, Oka Y, Shiota N, Kawamoto T, Song K, Miyazaki M. Marked species-difference in the vascular angiotensin II-forming pathways: humans versus rodents. Jpn J Pharmacol 1993; 62(2): 207-10.

44. Hollenberg N K. Implications of species differences for clinical investigation. Hypertension 2000; 35: 150-4.

45. Wolny A, Clozel J P, Rein J et al. Functional and biochemical analysis of angiotensin II-forming pathways in the human heart. Circulation Res 1997; 80(2): 219-27.

46. Liao Y, Husain A. The chymase-angiotensin system in humans: biochemistry molecular biology and potential role in cardiovascular diseases. Can J Cardiol 1995; 11(Suppl F): 13F-9F.

47. Urata H, Healy B, Stewart R W, Bumpus F M, Husain A. Angiotensin-II forming pathways in normal and failing hearts. Circ Res 1990; 66: 883-90.

48. Murakami M, Matsuda H, Kubota E, Wakino S, Honda M, Hayashi K, Saruta T. Role of angiotensin II generated by angiotensin-converting enzyme-independent pathways in canine kidney. Kidney Int 1997; 52(Suppl 63): S132-5.

49. Weir M R, Dzau V J. The renin-angiotensin-aldosterone system: a specific target for hypertension management. Am J Hypertens 1999; 12(12; Pt. 3): S205-13.

50. Rousseau M F, Konstam M A, Benedict et al. Progression of left ventricular dysfunction secondary to coronary artery disease sustained neurohormonal activation and effects of ibopamine therapy during long-term therapy with angiotensin-converting enzyme inhibitor. Am J Cardiol 1994; 73: 488-93.

51. Califf R M, Cohn J N. Cardiac protection: evolving role of angiotensin receptor blockers. Am Heart J 2000; 139(1 Pt 2): 15-22.

52. Ihara M, Urata H, Kinoshita A et al. Increased chymase-dependent angiotensin II formation in human atherosclerotic aorta. Hypertension 1999; 33(6): 1399-1405.

53. MacFadyen R J, Lees K R, Reid J L. Tissue and plasma angiotensin-converting enzyme and the response to ACE inhibitor drugs. Br J Clin Pharmacol 1991; 31(1): 1-13.

54. Ingert C, Grima M, Michel B, Barthelmebs M, Imbs J L. Renal tissue angiotensins during converting enzyme inhibition of angiotensin I in spontaneously hypertensive rat. Archiv Malad Coeur Vaisseaux 1997; 90(8): 1135-41.

55. Anderson S. Role of local and systemic angiotensin in diabetic renal disease. Kidney Int 1997; 52(Suppl 63): S107-10.

56. Jover B, Mimran A. Angiotensin II receptor antagonists versus angiotensin-converting enzyme inhibitors: effects on renal function. J Hypertens 1994; 12(Suppl 9): S3-9.

57. Braam B, Koomans H A. Renal responses to antagonism of the renin-angiotensin system. Curr Opin Nephrol Hypertens 1996; 5: 89-96.

58. Peters H, Border W A, Noble N A. Targeting TGF-beta over-expression in renal disease: Maximizing the antifibrotic action of angiotensin II blockade. Kidney Int 1998; 54(5): 1570-80.

59. Nakamura T, Obata J, Kimura H, Ohno S, Yoshida Y, Kawachi H, Shimizu F. Blocking angiotensin II ameliorates proteinuria and glomerular lesions in progressive mesangioproliferative glomerulonephritis. Kidney Int 1999; 55(3): 877-89.

60. Tazawa S, Nakane T, Chiba S. Angiotensin II type 1 receptor blockade prevents up-regulation of angiotensin II type 1A receptors in rat injured artery. J Pharmacol Exp Therap 1999; 288(2): 898-904.

61. Sachinidis A, Elhaschimi K, Ko Y, Seul C, Dusing R, Vetter H. CV-1 1974, the active metabolite of TCV-116 (candesartan) inhibits the synergistic or additive effect of different growth-factors on angiotensin-lI-induced proliferation of vascular smooth-muscle cells. Biochem Pharmacol 1996; 52(1): 123-6.

62. Zandi-Nejad K, Kusaka M, Taal M W, Ziai F, Kato S, Brenner B M, Mackenzie H S. Effects of candesartan and enalapril on cytokine expression in remnant kidney of munich-wistar (mw) rats 3 months after 5/6 nephrectomy (NPX). J Am Soc Nephrol 1998; 9: 626A.

63. Csikos T, Chung O, Unger T. Receptors and their classification: focus on angiotensin II and the AT2 receptor. J Hum Hypertens 1998; 12(5): 311-8.

64. Chung 0, Kuhl H, Stoll M, Unger T. Physiological and pharmacological implications of versus AT2 receptors. Kidney Int 1998; 54(Suppl 67): S95-9.

65. Maruyama K, Horiuchi M, Dzau V J, Takahashi H, Iwasaka T, Inada M, Matsubara H. Cardiac-specific overexpression of angiotensin II AT2 receptor causes attenuated response to ATi receptor-mediated pressor and chronotropic effects. J Clin Invest 1998; 101(3): 527-35.

66. Tsutsumi Y, Matsubara H, Masaki H et al. Angiotensin II type 2 receptor overexpression activates the vascular kinin system and causes vasodilation. J Clin Invest 1999; 104(7): 925-35.

67. Siragy H M, Carey R M. The subtype 2 (AT2) angiotensin receptor mediates renal production of nitric oxide in conscious rats. J Clin Invest 1997; 100(2): 264-9.

68. Jalowy A, Schulz R, Dorge H, Behrends M, Heusch G. Infarct size reduction by AT1-receptor blockade through a signal cascade of AT2-receptor activation bradykinin and prostaglandins in pigs. J Am Coll Cardiol 1998; 32(6): 1787-96.

69. Cohn J N, Tognoni G, Glazer R D, Spormann D, Hester A. Rationale and design of the Valsartan Heart Failure Trial: a large multinational trial to assess the effects of valsartan an angiotensin-receptor blocker on morbidity and mortality in chronic congestive heart failure. J Card Fail 1999;5(2): 155-60.

70. Mazzolai L, Maillard M, Rossat J, Nussberger J, Brunner H R, Bumier M. Angiotensin II receptor blockade in nornotensive subjects: a direct comparison of three AT1 receptor antagonists. Hypertension 1999; 33(3): 850-5.

71. Brunner-La Rocca H P, Vaddadi G, Esler M D. Recent insight into therapy of congestive heart failure: focus on ACE Inhibition and Angiotensin-II antagonism. J Am Coll Cardiol 1999; 33: 1163-73.

72. Messerli F. Editorial . . . And losartan was no better than placebo. J Hum Hypertens 1999; 13: 649-50.

73. Vanderheyden P M L, Fierens F L P, DeBacker J P, Fraeyman N, Vauquelin G. Distinction between surmountable and insurmountable selective AT receptor antagonists by use of CHO-KI cells expressing human angiotensin II AT receptors. Br J Pharmacol 1999; 126: 1057-65.

74. Brown N J, Agirbasli M, Vaughan D E. Comparative effect of angiotensin-converting enzyme inhibition and angiotensin II type 1 receptor antagonism on plasma fibrinolytic balance in humans. Hypertension 1999; 34(2): 285-90.

75. Kerins D M, Hao Q, Vaughan D E. Angiotensin induction of PAI-1 expression in endothelial cells is mediated by the hexapeptide angiotensin IV. J Clin Invest 1995; 96(5): 2515-20.

76. Ridker P M, Gaboury C L, Conlin P R, Seely E W, Williams G H, Vaughan D E. Stimulation of plasminogen activator inhibitor in vivo by infusion of angiotensin II. Evidence of a potential interaction between the renin-angiotensin system and fibrinolytic function. Circulation 1993; 87(6): 1969-73.

77. Goodfield N E R, Newby D E, Ludlam C A, Flapan A D. Effects of acute angiotensin II type 1 receptor antagonism and angiotensin-converting enzyme inhibition on plasma fibrinolytic parameters in patients with heart failure. Circulation 1999; 99: 2983-5.

78. Nishimura H, Tsuji H, Masuda H et al. Angiotensin II increases plasminogen activator inhibitor-1 and tissue factor mRNA expression without changing that of tissue type plasminogen activator or tissue factor pathway inhibitor in cultured rat aortic endothelial cells. Thromb Haemost 1997; 77(6): 1189-95.

79. Shihab F S, Bennett W M, Tanner A M, Andoh T F. Angiotensin II blockade decreases TGF-betal and matrix proteins in cyclosporine nephropathy. Kidney Int 1997; 52(3): 660-73.

80. Timmermans P B. Angiotensin II receptor antagonists: an emerging new class of cardiovascular therapeutics. Hypertens Res 1999; 22: 147-53.

81. Dzau V J. Tissue renin-angiotensin system in myocardial hypertrophy and failure. Arch Intern Med 1993; 153(8): 937-42.

82. Unger T, Ganten D, Lang R E, Scholkens B A. Is tissue converting enzyme inhibition a determinant of the antihypertensive efficacy of converting enzyme inhibitors? Studies with the two different compounds Hoe498 and MK421 in spontaneously hypertensive rats. J Cardiovasc Pharmacol 1984; 6: 872-80.

83. Cashin-Hemphill L, Holmvang G, Chan R C, Pitt B, Dinsmore R E, Lees R S. Angiotensin-converting enzyme inhibition as antiatherosclerotic therapy: no answer yet. QUIET Investigators. QUinapril Ischemic Event Trial. Am J Cardiol 1999; 83(1): 43-7.

84. Faxon D P. Effect of high dose angiotensin-converting enzyme inhibition on restenosis: final results of the MERCATOR study, a multicenter, double-blind, placebo-controlled trial of cilazipril. J Am Coll Cardiol 1995; 25: 362-9.

85. MERCATOR Study Group. Results of the Multicenter European Research trial with Cilazapril after Angioplasty to prevent Transluminal coronary Obstruction and Restenosis (MERCATOR) study: a multicenter randomized double-blind placebo-controlled trial. Circulation 1992; 86: 100-10.

86. Linz W, Wiemer G, Gohlke P, Unger T, Scholkens B A. Contribution of kinins to the cardiovascular actions of angiotensin-converting enzyme inhibitors. Pharmacol Rev 1995; 47(1): 25-49.

87. Carretero O A, Scicli A G. The kallikrein-kinin system as a regulator of cardiovascular and renal function in Laragh J H Brenner B M (eds): Hypertension: Pathophysiology Diagnosis and Management (2nd ed). New York, Raven Press 1995; 983-99.

88. Liu Y H, Yang X P, Sharov V G, Nass O, Sabbah H N, Peterson E, Carretero O A. Effects of angiotensin-converting enzyme inhibitors and angiotensin II type 1 receptor antagonists in rats with heart failure. J Clin Invest 1997; 99: 1926-35.

89. Gainer J V, Morrow J D, Loveland A, King D J, Brown N J. Effect of bradykinin-receptor blockade on the response to angiotensin-converting-enzyme inhibitor in normotensive and hypertensive subjects. New Engl J Med 1998; 339(18): 1285-92.

90. Hall J E. Control of blood pressure by the renin-angiotensin-aldosterone system. Clin Cardiol 1991; 14(Suppl IV): IV6-IV21.

91. Weinberg M S, Belknap S, Trebbin W, Solomon R J. Effects of changing salt and water balance on renal kallikrein kininogen and kinin. Kidney Int 1987; 31: 836-41.

92. Pitt B, Chang P, Grossman W, Dunlay M, Timmermans P B. Rationale background and design of the randomized angiotensin receptor antagonist—angiotensin-converting enzyme inhibitor study (RAAS). Am J Cardiol 1996; 78(10): 1129-31.

93. Ferrario C M, Chappell M C, Dean R H, Iyer S N. Novel angiotensin peptides regulate blood pressure endothelial function and natriuresis. J Am Soc Nephrol 1998; 9(9): 1716-22.

94. Iyer S N, Chappell M C, Averill D B, Diz D I, Ferrario C M. Vasodepressor actions of angiotensin-(1-7) unmasked during combined treatment with lisinopril and losartan. Hypertension 1998; 31(2): 699-705.

95. Ogihara T, Arakawa K. Clinical efficacy and tolerability of candesartan cilexetil. J Hum Hypertens 1999; 13(Suppl 1): S27-31.

96. Black H R, Graff A, Shute D, Stoltz R, Ruff D, Levine J, Shi Y, Mallows S. Valsartan a new angiotensin II antagonist for the treatment of essential hypertension; efficacy tolerability and safety compared to an angiotensin-converting enzyme inhibitor lisinopril. J Hum Hypertens 1997; 11(8): 483-9.

97. Bremner A D, Baur M, Oddou-Stock P, Bodin F. Valsartan: long-term efficacy and tolerability compared to lisinopril in elderly patients with essential hypertension. Clin Exp Hypertens 1997; 19(8): 1263-85.

98. Franke H. Antihypertensive effects of candesartan cilexetil enalapril and placebo. J Hum Hypertens 1997; 1 1(Suppl 2): S61-2.

99. Holwerda N J, Fogari R, Angeli P, Porcellati C, Hereng C, Oddou-Stock P, Heath R, Bodin F. Valsartan a new angiotensin II antagonist for the treatment of essential hypertension: efficacy and safety compared with placebo and enalapril. J Hypertens 1996; 14: 1147-51.

100. LaRochelle P, Flack J M, Marbury T C, Sareli P, Krieger E M, Reeves R A. Effects and tolerability of irbesartan versus enalapril in patients with severe hypertension. Am J Cardiol 1997; 80(12): 1613-5.

101. Mimran A, Ruilope L, Kerwin L, Nys M, Owens D, Kassler-Taub K, Osbakken M. A randomised double-blind comparison of the angiotensin II receptor antagonist irbesartan with the full-dose range of enalapril for the treatment of mild-to-moderate hypertension. J Hum Hypertens 1998; 12(3): 203-8.

102. Neutel J M, Frishman W H, Oparil S, Papademitriou V, Guthrie G. Comparison of telmisartan with lisinopril in patients with mild-to-moderate hypertension. Am J Ther 1999; 6(3): 161-6.

103. Pechere-Bertschi A, Nussberger J, Decosterd L et al. Renal responses to the angiotensin II receptor subtype 1 antagonist irbesartan versus enalapril in hypertensive patients. J Hypertens 1998; 16: 385-93.

104. Smith D H G, Neutel J M, Morgenstern P. Once-daily telmisartan compared with enalapril in the treatment of hypertension. Adv Ther 1998; 15(4): 229-40.

105. Townsend R, Haggert B, Liss C, Edelman J M. Efficacy and tolerability of losartan versus enalapril alone or in combination with hydrochlorothiazide in patients with essential hypertension. Clin Therap 1995; 17(5): 911-23.

106. Zanchetti A, Omboni S, DiBiagio C. Candesartan cilexetil and enalapril are of equivalent efficacy in patients with mild to moderate hypertension. J Hum Hypertens 1997; 11 (Suppl 2): S57-9.

107. Gradman A, Arcuri K E, Goldberg A I, Ikeda L S, Nelson E B, Snavely D B, Sweet C S. A randomized placebo-controlled double-blind parallel study of various doses of losartan potassium compared with enalapril maleate in patients with essential hypertension. Hypertension 1995; 25: 1345-50.

108. Tikkanen I, Omvik P, Jensen H A. Comparison of the angiotensin II antagonist losartan with the angiotensin converting enzyme inhibitor enalapril in patients with essential hypertension. J Hypertens 1995; 13(11): 1343-51.

109. Ruff D, Gazdick L P, Berman R, Goldberg A I, Sweet C S. Comparative effects of combination drug therapy regimens commencing with either losartan potassium an angiotensin II receptor antagonist or enalapril maleate for the treatment of severe hypertension. J Hypertens 1996; 14(2): 263-70.

110. Mallion J M, Siche J P, Lacourciere Y, Telmisartan Blood Pressure Monitoring Group. ABPM comparison of the antihypertensive profiles of the selective angiotensin II receptor antagonists telmisartan and losartan in patients with mild-to-moderate hypertension. J Hum Hypertens 1999; 13(10): 657-64.

111. Andersson O K, Neldam S. The antihypertensive effect and tolerability of candesartan cilexetil a new generation angiotensin II antagonist in comparison with losartan. Blood Pressure 1998; 7(1): 53-9.

112. Fogari R, Zoppi A, Mugellini A, Preti P, Banderali A, Pesce R M, Vanasia A. Comparative efficacy of losartan and valsartan in mild-to-moderate hypertension: Results of 24-hour ambulatory blood pressure monitoring. Curr Therap Res 1999; 60(4): 195-206.

113. Gradman A, Lewin A, Bowling B T et al. Comparative effects of candesartan cilexitil and losartan in patients with systemic hypertension. Heart Dis 1999; 1: 52-7.

114. Kassler-Taub K, Littlejohn T, Elliott W, Ruddy T, Adler E. Comparative efficacy of two angiotensin II receptor antagonists irbesartan and losartan in mild-to-moderate hypertension. Am J Hypertens 1998; 11(4): 445-53.

115. Lacourciere Y, Asmar R, for the Candesartan/Losartan study investigators. A comparison of the efficacy and duration of action of candesartan cilexetil and losartan as assessed by clinic and ambulatory blood pressure after a missed dose in truly hypertensive patients. A placebo-controlled forced titration study. Am J Hypertens 1999; 12(12): 1181-7.

116. Oparil S, Guthrie R, Lewin A J, Marbury T, Reilly K, Triscari J, Witcher J A. An elective-titration study of the comparative effectiveness of two angiotensin II-receptor blockers irbesartan and losartan. Clin Ther 1998; 20(3): 398-409.

117. Morsing P, Adler G, Brandt-Eliasson U et al. Mechanistic differences of various AT1-receptor blockers in isolated vessels of different origin. Hypertension 1999; 33:1406-1413.

118. Azizi M, Guyene T T, Chatellier G, Menard J: Blood pressure effects of acute intravenous renin or oral angiotensin converting enzyme inhibition in essential hypertension. J Hypertens 1994; 12(4): 419-27.

119. Fisher N D, AIIan D, Kifor I, Gaboury C L, Williams G H, Moore T J, Hollenberg N K. Responses to converting enzyme and renin inhibition. Role of angiotensin II in humans. Hypertension 1994; 23(1): 44-51.

120. Lacour C, Cazaubon C, Roccon A, Segondy D, Wagnon J, Nisato D. Effects of a renin inhibitor SR 43845 and of captopril on blood pressure and plasma active renin in conscious sodium-replete macaca. J Hypertens 1989; 7(Suppl 2): S33-5.

121. Oldham A A, Arnstein M J, Major J S, Clough D P. In vivo comparison of the renin inhibitor H77 with the angiotensin-converting enzyme inhibitor captopril. J Cardiovasc Pharmacol 1984; 6(4): 672-7.

122. Kiowski W, Beermann J, Rickenbacher P, Haemmerli R, Thomas M, Burkart F, Meinertz T. Angiotensinergic versus nonangiotensinergic hemodynamic effects of converting enzyme inhibition in patients with chronic heart failure. Assessment by acute renin and converting enzyme inhibition. Circulation 1994; 90(6): 2748-56.

123. Mento P F, Holt W F, Murphy W R, Wilkes B M. Combined renin and converting enzyme inhibition in rats. Hypertension 1989; 13(6 Pt 2): 741-8.

124. Fossa A A, Weinberg L J, Barber R L, Rauch A L, Nocerini M R, Murphy W R, Swindell A C. Synergistic effect on reduction in blood pressure with coadministration of the renin inhibitor CP-80 794 and the angiotensin-converting enzyme inhibitor captopril. J Cardiovasc Pharmacol 1992; 20(1): 75-82.

125. AIIan D R, Hui K Y, Coletti C, Hollenberg N K. Renin vs. angiotensin-converting enzyme inhibition in the rat: consequences for plasma and renal tissue angiotensin. J Pharmacol Exp Therap 1997 283(2): 661-5.

126. Pals D T, DeGraaf G L, Couch S J, Brunden M N. Additive combination studies of captopril and ditekiren a renin inhibitor in non-human primates. Clin Exper Hypertens 1991; 13(3): 425-36.

127. Hollenberg N K. Pharmacologic interruption of the renin-angiotensin system and the kidney: differential responses to angiotensin-converting enzyme and renin inhibition. J Am Soc Nephrol 1999; 10(1; Suppl 11): S239-42.

128. Blumenfeld J D, Sealey J E, Mann S J et al. Beta-adrenergic receptor blockade as a therapeutic approach for suppressing the renin-angiotensin-aldosterone system in normotensive and hypertensive subjects. Am J Hypertens 1999; 12(5): 451-9.

129. Holmer S R, Hense H W, Danser A H, Mayer B, Riegger G A, Schunkert-H. Beta adrenergic blockers lower renin in patients treated with ACE inhibitors and diuretics. Heart 1998; 80(1): 45-8.

130. Erb K A, Essig J, Breithaupt K, Belz G G. Clinical pharmacodynamic studies with cilazapril and a combination of cilazapril and propranolol. Drugs 1991; 41 (Suppl 1): 11-7.

131. VanGriensven J M, Seibert-Grafe M, Schoemaker H C, Frolich M, Cohen A F. The pharmacokinetic and pharmacodynamic interactions of ramipril with propranolol. Eur J Clin Pharmacol 1993; 45(3): 255-60.

132. Belz G G, Essig J, Kleinbloesem C H, Hoogkamer J F, Wiegand U W, Wellstein A. Interactions between cilazapril and propranolol in man; plasma drug concentrations hormone and enzyme responses hemodynamics agonist dose-effect curves and baroreceptor reflex. Br J Clin Pharmacol 1988; 26(5): 547-56.

133. Belz G G, Essig J, Erb K, Breithaupt K, Hoogkamer J F, Kneer J, Kleinbloesem C H. Pharmacokinetic and pharmacodynamic interactions between the ACE inhibitor cilazapril and beta-adrenoceptor antagonist propranolol in healthy subjects and in hypertensive patients. Br J Clin Pharm 1989; 27(Suppl): S317-22.

134. Bursztyn M, Gavras I, Gourley L, DeSilva J, Whalen J, Gavras H. Effect of combination therapy with atenolol and the angiotensin-converting enzyme inhibitor benazepril. Clinical Ther 1994; 16(3): 429-36.

135. Franz I W, Behr U, Ketelhut, R. Resting and exercise blood pressure with atenolol enalapril and a low-dose combination. J Hypertens 1987; 5(3; Suppl): S37-341.

136. Huttunen M, Lampainen E, Lilja M, Ikaheimo M, Kontro J, Makynen P, Savolainen A. Which antihypertensive to add to a beta-blocker. ACE inhibitor or diuretic? J Hum Hypertens 1992; 6(2): 121-5.

137. MacGregor G A, Markandu N D, Banks R A, Bayliss J, Roulston J E, Jones J C. Captopril in essential hypertension; contrasting effects of adding hydrochlorothiazide or propranolol. Br Med J 1982; 284(6317): 693-6.

138. MacGregor G A, Markandu N D, Smith S J, Sagnella G A. Captopril: contrasting effects of adding hydrochlorothiazide propranolol or nifedipine. J Cardiovasc Pharnacol 1985; 7(Suppl 1): S82-287.

139. Pickering T G, Case D B, Sullivan P A, Laragh J H. Comparison of antihypertensive and hormonal effects of captopril and propranolol at rest and during exercise. Am J Cardiol 1982; 49(6): 1566-8.

140. Soininen K, Gerlin-Pilra L, Sulhkonen J et al. A study of the effects of lisinopril when used in addition to atenolol. J Hum Hypertens 1992; 6(4): 321-4.

141. Staessen J, Fagard R, Lijnen P, Verschueren L J, Amery A. The hypotensive effect of propranolol in captopril-treated patients does not involve the plasma renin-angiotensin-aldosterone system. Clin Sci 1981; 61(Suppl 7): S441-4.

142. Staessen J, Fagard R, Lijnen P, Verschueren L J, Amery A. Double-blind comparison between propranolol and bendroflumethiazide in captopril-treated resistant hypertensive patients. Am Heart J 1983; 106(2): 321-8.

143. Swedish Lisinopril Study Group. Lisinopril combined with atenolol in the treatment of hypertension. J Cardiovasc Pharmacol 1991; 18(3): 457-61.

144. Wing L M, Chalmers J P, West M J et al. Enalapril and atenolol in essential hypertension: attenuation of hypotensive effects in combination. Clin Exp Hypertens 1988; 10(1): 119-33.

145. Manin-T-Veld A J. Vasodilation not cardiodepression underlies the antihypertensive effect of beta-adrenoceptor antagonists. Am J Cardiol 1991; 67(10): 13B-17B.

146. Fernandez H M, Leipzig R M, Larkin R J et al. Spironolactone in patients with heart failure. Correspondence. NEJM 2000; 342(2): 132-4.

147. McKelvie R S, Yusuf S, Pericak D et al. Comparison of Candesartan, enalapril and their combination in congestive heart failure: randomized evaluation of strategies for left ventricular dysfunction (RESOLVD) pilot study. Circulation 1999; 100: 1056-64.

148. Richer C, Bruneval P, Menard J, Giudicelli J F. Additive effects of enalapril and losartan in (mREN-2)27 transgenic rats. Hypertension 1998; 31(2): 692-8.

149. Azizi M, Guyene T T, Chatellier G, Wargon M, Menard J. Additive effects of losartan and enalapril on blood pressure and plasma active renin. Hypertension 1997; 29(2): 634-40.

150. Sommers D K, VanWyk M, Snyman J R. Additive effects of captopril and losartan on blood pressure in mildly sodium-depleted normotensives. Clin Drug Invest 1997; 14: 321-5.

151. Bentivoglio M, Buccolieri M, Carluccio E, Mariotti M, Politano M, Tommasi S, Corea, L. The association losartan-ramipril in essential hypertension. Am J Hypertens 1997; 10(4Pt2): 131A.

152. Koval S N, Maslyaeva L V, Penkova M Y, Bilovil A N, Starchenko T G. Advantages of combined use of losartan and enalapril in patients with mild arterial hypertension. Am J Hypertens 1999; 12(4): 137A.

153. Fogari R, Zoppi A, Corradi L et al. Adding losartan to lisinopril therapy in patients with hypertension: Assessment by 24-hour ambulatory blood pressure monitoring. Curr Therap Res 1999; 60(6): 326-34.

154. Zoppi A, Lazzari P, Preti P, Mugellini A, Lusardi P, Corradi L, Fogari R. Effect of lisinopril-losartan combination on insulin resistance in overweight hypertensive patients. Am J Hypertens 1998; 11(4 Pt 2): 113A.

155. Stergiou G S, Skeva I I, Baibas N M, Roussias L G, Kalkana C B, Achimastos A D, Mountokalakis T D. Additive hypotensive effect of angiotensin-converting enzyme inhibition and angiotensin-receptor antagonism in essential hypertension. J Cardiovasc Pharmacol 2000: 35(6): 937-41.

156. Ruilope L M, Aldigier J C, Ponticelli C, Oddou-Stock P, Botteri F, Mann J F on behalf of the European Group for the Investigation of Valsartan in Chronic Renal Disease. Safety of the combination of valsartan and benazepril in patients with chronic renal disease. J Hypertens 2000; 18: 89-95.

157. Cooper M E, Mogensen C E for the CALM study group. Role of candesartan, lisinopril and their combination on blood pressure and albuminuria in hypertensive microalbuminuric diabetic subjects. Am J Hypertens 2000; 13(4, Pt. 2): 59A.

158. Hamroff G, Blaufarb I, Mancini D, Katz S D, Bijou R, Jondeau G, Olivari M T, Thomas S, LeJemtel T H. Angiotensin II-receptor blockade further reduces afterload safely in patients maximally treated with angiotensin-converting enzyme inhibitors for heart failure. J Cardiovasc Pharmacol 1997; 30(4): 533-6.

159. Di Pasquale P, Bucca V, Scalzo S, Paterna S. Safety tolerability and neurohormonal changes of the combination captopril plus losartan in the early post-infarction period: a pilot study. Cardiovasc Drugs Ther 1998; 12: 211-6.

160. Di Pasquale P, Buccaa V, Scalzoa S, Cannizzaroa S, Giubilatoa A, Paternab S. Does the addition of losartan improve the beneficial effects of ACE inhibitors in patients with anterior myocardial infarction? A pilot study. Heart 1999; 81: 606-11.

161. Riegger G A, Bouzo H, Petr P et al. Improvement in exercise tolerance and symptoms of congestive heart failure during treatment with candesartan cilexetil. Symptom, Tolerability, Response to Exercise Trial of Candesartan Cilexetil in Heart Failure (STRETCH) Investigators. Circulation 1999; 100(22): 2224-30.

162. Gradman A H, Flanagan T L, Michelson E L. Tolerability and safety of high dose angiotensin II receptor blockade with candesartan cilexetil in combination with angiotensin-converting enzyme inhibition in chronic heart failure. Circulation 1999; 100(18; Suppl. I): 1783.

163. Sever P S, Holzgreve H. Long term efficacy and tolerability of candesartan cilexetil in patients with mild to moderate hypertension. J Hum Hypertens 1999; 13(Suppl 1): S69-73 1997.

164. Simko F, Simko J. Heart failure and angiotensin-converting enzyme inhibition: problems and perspectives. Physiol Res 1999; 48(1): 1-8.

165. Hamroff G, Katz S D, Mancini D et al. Addition of angiotensin II receptor blockade to maximal angiotensin-converting enzyme inhibition improves exercise capacity in patients with severe congestive heart failure. Circulation 1999; 99(8): 990-2.

166. Tonkon M, Awan N, Niazi I et al. Irbesartan combined with conventional therapy including angiotensin-converting enzyme inhibitors in heart failure. J Am Coll Cardiol 1998; 31(2):A188.

167. Luzier A B, DiTusa L. Underutilization of ACE inhibitors in heart failure. Pharmacotherapy 1999; 19(11): 1296-307.

168. Tanaka W, Flynn M, Sharma D et al. Plasma angiotensin II (AII) levels in heart failure patients: Effect of losartan (L) added to enalapril (E). Clin Pharmacol Therap 1998; 63(2): 177.

169. Guazzi M, Palermo P, Pontone G, Susini F, Agostoni P. Synergistic efficacy of enalapril and losartan on exercise performance and oxygen consumption at peak exercise in congestive heart failure. Am J Cardiol 1999; 84: 1038-43.

170. Spinale F G, deGasparo M, Whitebread S et al. Modulation of the renin-angiotensin pathway through enzyme inhibition and specific receptor blockade in pacing-induced heart failure: I. Effects on left ventricular performance and neurohormonal systems. Circulation 1997; 96(7): 2385-96.

171. Krombach R S, Clair M J, Hendrick J W et al. Angiotensin-converting enzyme inhibition AT1 receptor inhibition and combination therapy with pacing induced heart failure: effects on left ventricular performance and regional blood flow patterns. CardiovascRes 1998; 38(3): 631-45.

172. Ogilvie R I, Zborowska S D. Captopril and angiotensin II receptor antagonist therapy in a pacing model of heart failure. Can J Cardiol 1998; 14(8): 1025-33.

173. Shen Y T, Wiedmann R T, Greenland B D, Lynch J J, Grossman W. Combined effects of angiotensin-converting enzyme inhibition and angiotensin II receptor antagonism in conscious pigs with congestive heart failure. Cardiovasc Res 1998; 39(2): 413-22.

174. Spinale F G, lannini J P, Mukherjee R, Melton D M, deGasparo M. Angiotensin AT1 receptor inhibition angiotensin-converting enzyme inhibition and combination therapy with developing heart failure: cellular mechanisms of action. J Card Failure 1998; (4):325-32.

175. Spinale F G, Mukherjee R, lannini J P et al. Modulation of the renin-angiotensin pathway through enzyme inhibition and specific receptor blockade in pacing-induced heart failure: II. Effects on myocyte contractile processes. Circulation 1997; 96(7): 2397-406.

176. Tocchi M, Rosanio S, Anzuini A, Sposi A, Mattioli D. Angiotensin II receptor blockade combined to ACE-inhibition improves left ventricular dilation and exercise ejection fraction in congestive heart failure. J Am Coll Cardiol 1998; 31(2): A188.

177. Nunez E, Hosoya K, Susic D, Frohlich E D. Enalapril and losartan reduced cardiac mass and improved coronary hemodynamics in SHR. Hypertension 1997; 29(1 Pt 2): 519-24.

178. Taylor K, Patten R D, Smith J J, Aronovitz M J, Wight J, Salomon R N, Konstam M A. Divergent effects of angiotensin-converting enzyme inhibition and angiotensin II-receptor antagonism on myocardial cellular proliferation and collagen deposition after myocardial infarction in rats. J Cardiovasc Pharmacol 1998; 31(5): 654-60.

179. Pfeffer M A. Enhancing cardiac protection after myocardial infarction: Rationale for newer clinical trials of angiotensin receptor blockers. Am Heart J 2000; 139(1 pt 2): 23-8.

180. Swedberg K, Pfeffer M, Granger C et al. Candesartan in heart failure—assessment of reduction in mortality and morbidity (CHARM): rationale and design. J Card Fail 1999; 5(3): 276-82.

181. Pitt B, Dickstein K, Benedict C R et al. The Randomized Angiotensin Receptor Antagonist—ACE-Inhibitor Study (RAAS)—Pilot-Study. Circulation 1996; 94(8): 12497.

182. Kasiske B L, Kalil R S, Ma J Z, Liao M, Keane W F. Effect of anti-hypertensive therapy on the kidney in patients with diabetes: a meta-regression analysis. Ann Intern Med 1993; 118(2): 129-38.

183. Ritz E. Nephropathy in patients with type 2 diabetes mellitus. N Engl J Med 1999; 341(15):1127-33.

184. DeJong P E, deZeeuw D. Renoprotective therapy: titration against urinary protein excretion. Lancet 1999; 354(9176): 352-3.

185. Andersen S, Tamow L, Rossing P, Hansen B V, Parving H H. Renoprotective effects of angiotensin II receptor blockade in type 1 diabetic patients with diabetic nephropathy. Kidney Int 2000; 57(2): 601-6.

186. Mackenzie H S, Ziai F, Omer S A, Nadim M K, Taal M W. Angiotensin receptor blockers in chronic renal disease: the promise of a bright clinical future. J Am Soc Nephrol 1999; 10(Suppl 12): S283-6.

187. Taal M W, Brenner B M. Renoprotective benefits of RAS inhibition: From ACEI to angiotensin II antagonists. Kidney Int 2000; 57: 1803-17.

188. Burdmann, E A, Andoh T F, Nast C C et al. Prevention of experimental cyclosporine-induced interstitial fibrosis by losartan and enalapril. Am J Physiol 1995; 38(4): 491-9.

189. Ots M, Mackenzie H S, Troy J L, Rennke H G, Brenner B M. Effects of combination therapy with enalapril and losartan on the rate of progression of renal injury in rats with 5/6 renal mass ablation. J Am Soc Nephrol 1998; 9(2): 224-30.

190. Weir M R, Dworkin L D. Antihypertensive drugs dietary salt and renal protection: how low should you go and with which therapy? Am J Kidney Dis 1998; 32(1): 1-22.

191. Akasu M, Urata H, Kinoshita A, Sasaguri M, Ideishi M, Arakawa K. Differences in tissue angiotensin II-forming pathways by species and organs in vitro. Hypertension 1998; 32(3): 514-20.

192. Pitt B, Segal R, Martinez F A et al. Randomised trial of losartan versus captopril in patients over 65 with heart failure (Evaluation of Losartan in the Elderly Study ELITE). Lancet 1997; 349(9054): 747-52.

193. Schmitt F, Natov S, Martinez F, Lacour B, Hannedouche T P. Renal effects of angiotensin I-receptor blockade and angiotensin convertase inhibition in man. Clin Sci 1996; 90(3): 205-13.

194. Russo D, Pisani A, Balletta M, De Nicola L, Savino F A, Andreucci M, Minutolo R. Additive antiproteinuric effect of converting enzyme inhibitor and losartan in normotensive patients with IgA nephropathy. Am J Kidney Dis 1999; 33(5): 851-6.

195. Arteaga J, Petrina E, Anda E, Calderon D, Sorbet M, Asirón M. The antiproteinuric effect of enalapril is potentiated by losartan in normotensive patients with diabetic nephropathy. Am J Hypertens 2000; 13(4; Pt. 2): 117A.

196. Geiger H. Are angiotensin II receptor blockers superior to angiotensin-converting enzyme inhibitors with regard to their renoprotective effect? Nephrol Dial Transplant 1997; 12(4): 640-2.

197. Jover B, Casellas D, Mimran A, Nisato D, Cazaubon C. Renal effects of combined treatment by irbesartan and enalapril in L-NAME hypertension in rats. Naunyn Schmied Archiv Pharmacol 1998; 358(Suppl 1): 3825.

198. Bakris G L, Siomos M, Bolton W K, Hebert L, Agerwall R, Catanzaro D, Janssen I. Differential Effects of Valsartan (V) and Lisinopril (L) on Potassium [K+] Homeostasis in Hypertensive Patients with Nephropathy. J Am Soc Nephrol 1999; 10(9): A0349.

199. Heart Outcomes Prevention Evaluation (HOPE) Study Investigators. Effects of an angiotensin-converting-enzyme inhibitor ramipril on death from cardiovascular causes myocardial infarction and stroke in high-risk patients. N Engl J Med 2000; 342(3): 145-53.

200. Mogensen C E et al., Randomised controlled trial of dual blockade of renin-angotensin system in patients with hypertension, microalbuminuria, and non-insulin dependen diabetes: the candesartan and lisinopril microalbuminuria (CALM) study. BMJ 2000; 321: 1440-4.

While the invention has been described with respect to certain embodiments, it should be appreciated that many modifications and changes may be made by those of ordinary skill in the art without departing from the spirit of the invention. It is intended that such modifications, changes, and equivalents fall within the scope of the following claims.

What is claimed is:

1. A method for treating an angiotensin II (AII)-mediated tissue effect in a subject, comprising:
administering to a subject in need of treatment for an AII-mediated tissue effect an amount of an angiotensin II receptor blocker (ARB) effective for reducing an AII-mediated tissue effect in the subject, wherein the amount of ARB effective for reducing the AII-mediated tissue effect in the subject is more than an amount of the ARB that is effective for reducing or controlling blood pressure of the subject.

2. The method of claim 1, wherein the amount of ARB effective for reducing an AII-mediated tissue effect is at least one-and-a-half times an amount effective for treatment or control of hypertension in the subject.

3. The method of claim 1, wherein the amount of ARB effective for reducing an AII-mediated tissue effect is at least three times an amount effective for treatment or control of hypertension in the subject.

4. The method of claim 1, wherein the amount of ARB effective for reducing an AII-mediated tissue effect is about three times to about twenty times an amount effective for treatment or control of hypertension in the subject.

5. The method of claim 1, wherein the amount of ARB effective for reducing an AII-mediated tissue effect is at least one-and-a-half times a maximum daily dose recommended or approved for treatment or control of hypertension.

6. The method of claim 1, wherein the amount of ARB effective for reducing an AII-mediated tissue effect is at least three times a maximum daily dose recommended or approved for treatment or control of hypertension.

7. The method of claim 1, wherein the amount of ARB effective for reducing an AII-mediated tissue effect is about three times to about twenty times a maximum daily dose recommended or approved for treatment or control of hypertension.

8. The method of claim 1, wherein the AII-mediated tissue effect is an aneurysm.

9. The method of claim 8, wherein the aneurysm is an aortic aneurysm.

10. The method of claim 8, wherein the aneurysm is an aortic root aneurysm.

11. The method of claim 1, wherein the AII-mediated tissue effect is proteinuria.

12. The method of claim 1, wherein the AII-mediated tissue effect is microalbuminuria.

13. The method of claim 1, wherein the AII-mediated tissue effect is chronic or congestive heart failure (CHF).

14. The method of claim 1, wherein the AII-mediated tissue effect is atherogenesis.

15. The method of claim 1, wherein the AII-mediated tissue effect is atherosclerosis.

16. The method of claim 15, wherein the atherosclerosis is associated with at least one condition selected from scleroderma, lupus erythematosus, rheumatoid arthritis, kidney disease, and solid organ transplantation.

17. The method of claim 1, wherein the AII-mediated tissue effect is tissue hypertrophy.

18. The method of claim 17, wherein the tissue hypertrophy is vascular tissue hypertrophy.

19. The method of claim 1, wherein the AII-mediated tissue effect is cytokine production.

20. The method of claim 19, wherein the cytokine is transforming growth factor beta (TGF-β).

21. The method of claim 1, wherein the administering involves a plurality of ARBs.

22. The method of claim 1, wherein the ARB is at least one compound selected from candesartan, eprosartan, irbesartan, losartan, olmesartan, telmisartan, valsartan, and prodrugs and salts thereof.

23. The method of claim 1, wherein the ARB is at least one compound selected from candesartan, irbesartan, and prodrugs and salts thereof.

24. The method of claim 1, wherein the ARB is candesartan cilexetil.

25. The method of claim 1, further comprising administering to the subject at least one compound selected from aspirin, beta-blockers, aldactone, and compounds that can inhibit atherogenesis or platelet adhesion.

26. The method of claim 1, further comprising administering to the subject at least one additional agent selected from diuretics, peripheral adrenergic blockers, central adrenergic stimulants, calcium channel blockers, vasodilators, and other antihypertensive agents excluding angiotensin converting enzyme inhibitors (ACEI).

27. A method for treating an AII-mediated tissue effect in a normotensive subject, comprising:
administering to a subject in need of treatment for an AII-mediated tissue effect an amount of an ARB effective for reducing an AII-mediated tissue effect in the subject, wherein the subject does not have hypertension, and wherein the amount of ARB effective for reducing an AII-mediated tissue effect in the subject is more than an amount of the ARB that is usually effective for reducing or controlling blood pressure in hypertensive subjects.

28. The method of claim 27, wherein the amount of ARB effective for reducing an AII-mediated tissue effect is at least one-and-a-half times a maximum daily dose recommended or approved for treatment or control of hypertension.

29. The method of claim 27, wherein the amount of ARB effective for reducing an AII-mediated tissue effect is at least three times a maximum daily dose recommended or approved for treatment or control of hypertension.

30. The method of claim 27, wherein the amount of ARB effective for reducing an AII-mediated tissue effect is about three times to about twenty times a maximum daily dose recommended or approved for treatment or control of hypertension.

31. The method of claim 27, wherein the AII-mediated tissue effect is an aneurysm.

32. The method of claim 31, wherein the aneurysm is an aortic aneurysm.

33. The method of claim 31, wherein the aneurysm is an aortic root aneurysm.

34. The method of claim 27, wherein the AII-mediated tissue effect is proteinuria.

35. The method of claim 27, wherein the AII-mediated tissue effect is microalbuminuria.

36. The method of claim 27, wherein the AII-mediated tissue effect is CHF.

37. The method of claim 27, wherein the AII-mediated tissue effect is atherogenesis.

38. The method of claim 27, wherein the AII-mediated tissue effect is atherosclerosis.

39. The method of claim 38, wherein the atherosclerosis is associated with at least one condition selected from scleroderma, lupus erythematosus, rheumatoid arthritis, kidney disease, and solid organ transplantation.

40. The method of claim 27, wherein the AII-mediated tissue effect is tissue hypertrophy.

41. The method of claim 40, wherein the tissue hypertrophy is vascular tissue hypertrophy.

42. The method of claim 27, wherein the AII-mediated tissue effect is cytokine production.

43. The method of claim 42, wherein the cytokine is TGF-β.

44. The method of claim 27, wherein the administering involves a plurality of ARBs.

45. The method of claim 27, wherein the ARB is at least one compound selected from candesartan, eprosartan, irbesartan, losartan, olmesartan, telmisartan, valsartan, and prodrugs and salts thereof.

46. The method of claim 27, wherein the ARB is at least one compound selected from candesartan, irbesartan, and prodrugs and salts thereof.

47. The method of claim 27, wherein the ARB is candesartan cilexetil.

48. The method of claim 27, further comprising administering to the subject at least one compound selected from aspirin, beta-blockers, aldactone, and compounds that can inhibit atherogenesis or platelet adhesion.

49. The method of claim 27, further comprising administering to the subject at least one additional agent selected from diuretics, peripheral adrenergic blockers, central adrenergic stimulants, calcium channel blockers, vasodilators, and other antihypertensive agents excluding ACEIs.

50. A method for treating an AII-mediated tissue effect in a subject, comprising:
administering to a subject in need of treatment for an AII-mediated tissue effect an amount of ARB, and
administering to the subject an amount of ACEI, wherein the amount of ARB and the amount of ACEI together is (1) effective for reducing the AII-mediated tissue effect in the subject and (2) more than an amount effective for achieving essentially a same degree of blood pressure reduction or blood pressure control in the subject.

51. The method of claim 50, wherein the amount of ARB is more than an effective amount for achieving essentially the same degree of blood pressure reduction or blood pressure control in the subject.

52. The method of claim 50, wherein the amount of ARB is at least one-and-a-half times an amount effective for treatment or control of hypertension in the subject.

53. The method of claim 50, wherein the amount of ARB is at least three times an amount effective for treatment or control of hypertension in the subject.

54. The method of claim 50, wherein the amount of ARB is about three times to about twenty times an amount effective for treatment or control of hypertension in the subject.

55. The method of claim 50, wherein the amount of ARB is at least one-and-a-half times a maximum daily dose recommended or approved for treatment or control of hypertension.

56. The method of claim 50, wherein the amount of ARB is at least three times a maximum daily dose recommended or approved for treatment or control of hypertension.

57. The method of claim 50, wherein the amount of ARB is about three times to about twenty times a maximum daily dose recommended or approved for treatment or control of hypertension.

58. The method of claim 50, wherein the AII-mediated tissue effect is an aneurysm.

59. The method of claim 58, wherein the aneurysm is an aortic aneurysm.

60. The method of claim 58, wherein the aneurysm is an aortic root aneurysm.

61. The method of claim 50, wherein the AII-mediated tissue effect is proteinuria.

62. The method of claim 50, wherein the AII-mediated tissue effect is microalbuminuria.

63. The method of claim 50, wherein the AII-mediated tissue effect is CHF.

64. The method of claim 50, wherein the AII-mediated tissue effect is atherogenesis.

65. The method of claim 50, wherein the AII-mediated tissue effect is atherosclerosis.

66. The method of claim 65, wherein the atherosclerosis is associated with at least one condition selected from scleroderma, lupus erythematosus, rheumatoid arthritis, kidney disease, and solid organ transplantation.

67. The method of claim 50, wherein the AII-mediated tissue effect is tissue hypertrophy.

68. The method of claim 67, wherein the tissue hypertrophy is vascular tissue hypertrophy.

69. The method of claim 50, wherein the AII-mediated tissue effect is cytokine production.

70. The method of claim 69, wherein the cytokine is TGF-β.

71. The method of claim 50, wherein the administering an amount of ARB involves a plurality of ARBs.

72. The method of claim 50, wherein the ARB is at least one compound selected from candesartan, eprosartan, irbesartan, losartan, olmesartan, telmisartan, valsartan, and prodrugs and salts thereof.

73. The method of claim 50, wherein the ARB is at least one compound selected from candesartan, irbesartan, and prodrugs and salts thereof.

74. The method of claim 50, wherein the ARB is candesartan cilexetil.

75. The method of claim 50, wherein the administering an amount of ACEI involves a plurality of ACEIs.

76. The method of claim 50, wherein the ACEI is at least one compound selected from benazepril, captopril, enalapril, fosinopril, lisinopril, moexipril, perindopril, quinapril, ramipril, trandolapril, and prodrugs and salts thereof.

77. The method of claim 50, further comprising administering to the subject at least one compound selected from aspirin, beta-blockers, aldactone, and compounds that can inhibit atherogenesis or platelet adhesion.

78. The method of claim 50, further comprising administering to the subject at least one additional agent selected from diuretics, peripheral adrenergic blockers, central adrenergic stimulants, calcium channel blockers, and vasodilators.

79. A method for treating an AII-mediated tissue effect in a normotensive subject, comprising:
administering to a subject in need of treatment for an AII-mediated tissue effect an amount of ARB, and
administering to the subject an amount of ACEI, wherein the subject does not have hypertension, and wherein the amount of ARB and the amount of ACEI together is (1) effective for reducing the AII-mediated tissue effect in the subject and (2) more than an amount that is usually effective for reducing or controlling blood pressure in hypertensive subjects.

80. The method of claim 79, wherein the amount of ARB is at least one-and-a-half times a maximum daily dose recommended or approved for treatment or control of hypertension.

81. The method of claim 79, wherein the amount of ARB is at least three times a maximum daily dose recommended or approved for treatment or control of hypertension.

82. The method of claim 79, wherein the amount of ARB is about three times to about twenty times a maximum daily dose recommended or approved for treatment or control of hypertension.

83. The method of claim 79, wherein the AII-mediated tissue effect is an aneurysm.

84. The method of claim 83, wherein the aneurysm is an aortic aneurysm.

85. The method of claim 83, wherein the aneurysm is an aortic root aneurysm.

86. The method of claim 79, wherein the AII-mediated tissue effect is proteinuria.

87. The method of claim 79, wherein the AII-mediated tissue effect is microalbuminuria.

88. The method of claim 79, wherein the AII-mediated tissue effect is CHF.

89. The method of claim 79, wherein the AII-mediated tissue effect is atherogenesis.

90. The method of claim 79, wherein the AII-mediated tissue effect is atherosclerosis.

91. The method of claim 90, wherein the atherosclerosis is associated with at least one condition selected from scleroderma, lupus erythematosus, rheumatoid arthritis, kidney disease, and solid organ transplantation.

92. The method of claim 79, wherein the AII-mediated tissue effect is tissue hypertrophy.

93. The method of claim 92, wherein the tissue hypertrophy is vascular tissue hypertrophy.

94. The method of claim 79, wherein the AII-mediated tissue effect is cytokine production.

95. The method of claim 94, wherein the cytokine is TGF-β.

96. The method of claim 79, wherein the administering an amount of ARB involves a plurality of ARBs.

97. The method of claim 79, wherein the ARB is at least one compound selected from candesartan, eprosartan, irbesartan, losartan, olmesartan, telmisartan, valsartan, and prodrugs and salts thereof.

98. The method of claim 79, wherein the ARB is at least one compound selected from candesartan, irbesartan, and prodrugs and salts thereof.

99. The method of claim 79, wherein the ARB is candesartan cilexetil.

100. The method of claim 79, wherein the administering an amount of ACEI involves a plurality of ACEIs.

101. The method of claim 79, wherein the ACEI is at least one compound selected from benazepril, captopril, enalapril, fosinopril, lisinopril, moexipril, perindopril, quinapril, ramipril, trandolapril, and prodrugs and salts thereof.

102. The method of claim 79, further comprising administering to the subject at least one compound selected from aspirin, beta-blockers, aldactone, and compounds that can inhibit atherogenesis or platelet adhesion.

103. The method of claim 79, further comprising administering to the subject at least one additional antihypertensive agent selected from diuretics, peripheral adrenergic blockers, central adrenergic stimulants, calcium channel blockers, and vasodilators.

104. A pharmaceutical composition for treating an AII-mediated tissue effect in a subject, comprising an amount of ARB and an amount of ACEI, wherein the amount of ARB and the amount of ACEI together is (1) effective for reducing an AII-mediated tissue effect in a subject and (2) more than an amount that is usually effective for reducing or controlling blood pressure in hypertensive subjects.

105. The pharmaceutical composition of claim 104, further comprising a pharmaceutically acceptable carrier.

106. The pharmaceutical composition of claim 104, wherein the amount of ARB is at least one-and-a-half times a maximum daily dose recommended or approved for treatment or control of hypertension.

107. The pharmaceutical composition of claim 104, wherein the amount of ARB is at least three times a maximum daily dose recommended or approved for treatment or control of hypertension.

108. The pharmaceutical composition of claim 104, wherein the amount of ARB is about three times to about twenty times a maximum daily dose recommended or approved for treatment or control of hypertension.

109. The pharmaceutical composition of claim 104, wherein the AII-mediated tissue effect is an aneurysm.

110. The pharmaceutical composition of claim 109, wherein the aneurysm is an aortic aneurysm.

111. The pharmaceutical composition of claim 109, wherein the aneurysm is an aortic root aneurysm.

112. The pharmaceutical composition of claim 104, wherein the AII-mediated tissue effect is proteinuria.

113. The pharmaceutical composition of claim 104, wherein the AII-mediated tissue effect is microalbuminuria.

114. The pharmaceutical composition of claim 104, wherein the A11-mediated tissue effect is CHF.

115. The pharmaceutical composition of claim 104, wherein the AII-mediated tissue effect is atherogenesis.

116. The pharmaceutical composition of claim 104, wherein the AII-mediated tissue effect is atherosclerosis.

117. The pharmaceutical composition of claim 116, wherein the atherosclerosis is associated with at least one condition selected from scleroderma, lupus erythematosus, rheumatoid arthritis, kidney disease, and solid organ transplantation.

118. The pharmaceutical composition of claim 104, wherein the AII-mediated tissue effect is tissue hypertrophy.

119. The pharmaceutical composition of claim 118, wherein the tissue hypertrophy is vascular tissue hypertrophy.

120. The pharmaceutical composition of claim 104, wherein the AII-mediated tissue effect is cytokine production.

121. The pharmaceutical composition of claim 120, wherein the cytokine is TGF-β.

122. The pharmaceutical composition of claim 104, wherein the amount of ARB involves a plurality of ARBs.

123. The pharmaceutical composition of claim 104, wherein the ARB is at least one compound selected from candesartan, eprosartan, irbesartan, losartan, olmesartan, telmisartan, valsartan, and prodrugs and salts thereof.

124. The pharmaceutical composition of claim 104, wherein the ARB is at least one compound selected from candesartan, irbesartan, and prodrugs and salts thereof.

125. The pharmaceutical composition of claim 104, wherein the ARB is candesartan cilexetil.

126. The pharmaceutical composition of claim 104, wherein the amount of ACEI involves a plurality of ACEIs.

127. The pharmaceutical composition of claim 104, wherein the ACEI is at least one compound selected from benazepril, captopril, enalapril, fosinopril, lisinopril, moexipril, perindopril, quinapril, ramipril, trandolapril, and prodrugs and salts thereof.

128. The pharmaceutical composition of claim 104, further comprising at least one compound selected from aspirin, beta-blockers, aldactone, and compounds that can inhibit atherogenesis or platelet adhesion.

129. The pharmaceutical composition of claim 104, further comprising at least one additional agent selected from diuretics, peripheral adrenergic blockers, central adrenergic stimulants, calcium channel blockers, and vasodilators.

130. The pharmaceutical composition of claim 104, wherein the pharmaceutical composition is formulated for oral administration.

131. The pharmaceutical composition of claim 130, wherein the pharmaceutical composition is formulated for once-daily administration.

132. The pharmaceutical composition of claim 131, wherein the pharmaceutical composition is formulated for twice-daily administration.

133. The pharmaceutical composition of claim 104, wherein the pharmaceutical composition is formulated for parenteral administration.
